SEQUENCE LISTING (26 sequences)

SEQ ID NO: 1 - protein, 8 aa, synthetic construct
GFTFSSYY

SEQ ID NO: 2 - protein, 8 aa, synthetic construct
INSQGGST

SEQ ID NO: 3 - protein, 12 aa, synthetic construct
ARSDYDWAWFAY

SEQ ID NO: 4 - protein, 12 aa, synthetic construct
QSLLYSSNQKNY

SEQ ID NO: 5 - protein, 9 aa, synthetic construct
QQYYRYPPT

SEQ ID NO: 6 - protein, 8 aa, synthetic construct
GFTFSSYM

SEQ ID NO: 7 - protein, 12 aa, synthetic construct
ARSDYDWAWYAY

SEQ ID NO: 8 - protein, 119 aa, synthetic construct
DVKLVESGGGLVKLGGSLKLSCAASGFTFSSYYMSWVRQTPEKRLELVAAINSQGGSTYYPDTVKGRFTISRDNAKNTLYLQMSSLKSEDTGLYYCARSDYDWAWFAYWGQGTLVTVSA

SEQ ID NO: 9 - protein, 113 aa, synthetic construct
DIVMSQSPSSLAVSVGEKVAMSCKSSQSLLYSSNQKNYLAWYQQKPGQSPKLLIYWASTRESGVPDRFTGSGSGTDFTLTISSVKAEDLAVYYCQQYYRYPPTFGAGTKLELK

SEQ ID NO: 10 - protein, 119 aa, synthetic construct
EVQLVESGGGLVQPGGSLRLSCAASGFTFSSYYMSWVRQAPGKRLELVAAINSQGGSTYYPDTVKGRFTISRDNAKNTLYLQMNSLRAEDTAVYYCARSDYDWAWFAYWGQGTLVTVSS

SEQ ID NO: 11 - protein, 113 aa, synthetic construct
DIVMTQSPSSLAVSLGERATINCKSSQSLLYSSNQKNYLAWYQQKPGQPPKLLIYWASTRESGVPDRFSGSGSGTDFTLTISSLQAEDVAVYYCQQYYRYPPTFGGGTKLEIK

SEQ ID NO: 12 - protein, 119 aa, synthetic construct
EVQLVESGGGLVQPGGSLRLSCAASGFTFSSYMMSWVRQAPGKRLELVAAINSQGGSTYYPDTVEGRFTISRDNAKNTLYLQMNSLRAEDTAVYYCARSDYDWAWYAYWGQGTLVTVSS

SEQ ID NO: 13 - protein, 113 aa, synthetic construct
DIVMTQSPSSLAVSLGERATINCKSSQSLLYSSNQKNYLAWYQQKPGQPPKLLIYWANTRESGVPDRFSGSGSGTDFTLTISSLQAEDVAVYYCQQYYRYPPTFGGGTKLEIK

SEQ ID NO: 14 - protein, 449 aa, synthetic construct
DVKLVESGGGLVKLGGSLKLSCAASGFTFSSYYMSWVRQTPEKRLELVAAINSQGGSTYYPDTVKGRFTISRDNAKNTLYLQMSSLKSEDTGLYYCARSDYDWAWFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 15 - protein, 220 aa, synthetic construct
DIVMSQSPSSLAVSVGEKVAMSCKSSQSLLYSSNQKNYLAWYQQKPGQSPKLLIYWASTRESGVPDRFTGSGSGTDFTLTISSVKAEDLAVYYCQQYYRYPPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 446 aa, synthetic construct
DVKLVESGGGLVKLGGSLKLSCAASGFTFSSYYMSWVRQTPEKRLELVAAINSQGGSTYYPDTVKGRFTISRDNAKNTLYLQMSSLKSEDTGLYYCARSDYDWAWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 220 aa, synthetic construct
DIVMSQSPSSLAVSVGEKVAMSCKSSQSLLYSSNQKNYLAWYQQKPGQSPKLLIYWASTRESGVPDRFTGSGSGTDFTLTISSVKAEDLAVYYCQQYYRYPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 446 aa, synthetic construct
EVQLVESGGGLVQPGGSLRLSCAASGFTFSSYYMSWVRQAPGKRLELVAAINSQGGSTYYPDTVKGRFTISRDNAKNTLYLQMNSLRAEDTAVYYCARSDYDWAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 220 aa, synthetic construct
DIVMTQSPSSLAVSLGERATINCKSSQSLLYSSNQKNYLAWYQQKPGQPPKLLIYWASTRESGVPDRFSGSGSGTDFTLTISSLQAEDVAVYYCQQYYRYPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 449 aa, synthetic construct
EVQLVESGGGLVQPGGSLRLSCAASGFTFSSYYMSWVRQAPGKRLELVAAINSQGGSTYYPDTVKGRFTISRDNAKNTLYLQMNSLRAEDTAVYYCARSDYDWAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 449 aa, synthetic construct
EVQLVESGGGLVQPGGSLRLSCAASGFTFSSYMMSWVRQAPGKRLELVAAINSQGGSTYYPDTVEGRFTISRDNAKNTLYLQMNSLRAEDTAVYYCARSDYDWAWYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 220 aa, synthetic construct
DIVMTQSPSSLAVSLGERATINCKSSQSLLYSSNQKNYLAWYQQKPGQPPKLLIYWANTRESGVPDRFSGSGSGTDFTLTISSLQAEDVAVYYCQQYYRYPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 538 aa, synthetic construct
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 24 - protein, 538 aa, synthetic construct
MARAVALLPSRSPPTPLLWPLLLLLLRKTGAQDVRVQVLPEVRGQLGGTVELPCHLLPPVPGLYISLVTWQRPDAPPDHQNVAAFHPKMGPSFPSPKPGSQRLSFVSAKQSTRQDTEAELQDATLALRGLTVEDEGNYTCEFATFPKGSVRGMTWLRVIAKPQNHAEAQEVTFSQDPVPVARCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVHSNPEPTGYDWSTTSGIFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEEQEMPSQLFTLGASEHSPLKTPYFDAGASCTEQEMPRYHELPTLEERSGPLHPGATSLGSPIPVPPGPPVVEDVSLDLEDEEGEEEEEYLDKINPVYDALSYSSPSDSYQGKGFVMSRAMYV

SEQ ID NO: 25 - DNA, 1614 bp, synthetic construct
  source 1..1614: mol_type = other DNA
atggcccgcgctgcagcccttttgccttcccgctctccccctacaccactcctgtggccactgcttctgctgctcctgcttgagacaggagcccaggacgtcagggtgcaggtgctgcctgaagtgaggggccagctggggggtacagtagagttgccatgccacctgctgccaccagtccctggcctgtacatatctctggtcacctggcagcgtcccgatgcacccgctaatcaccagaacgtggcagcctttcaccccaagatgggtccaagcttccctagtcccaaacctgggtcagagcggctctccttcgttagtgctaaacagagtacaggacaggacaccgaagccgaactgcaggacgctactctcgctcttcatggtttgacagtggaggacgaaggcaactacacctgtgagttcgctacatttcctaaaggttccgtgcggggtatgacctggttgagggtgattgcaaagcccaagaaccaggccgaggcccagaaagttacattttcacaggatcccactaccgtcgcactgtgcattagtaaggaggggaggccacctgcacgaatcagttggctgtccagccttgactgggaggccaaggaaacccaagttagcggaactctggctgggaccgtcacagtgacaagcaggtttactctcgtcccctcagggagagcagatggtgtgacagtgacatgtaaggttgagcatgaatcctttgaggagccagccttgattcccgttactttgagcgtccggtatcctcccgaggtgtcaatttcaggctatgatgacaactggtatctcggtcggacagatgccacactgagctgtgatgtgcgaagtaaccccgaacctaccggctatgactggagtaccacatctggtaccttcccaacctctgccgtggcacagggcagccagctcgtcatacatgccgttgactcactgttcaacactacatttgtgtgcactgtgactaacgccgtgggaatgggacgggccgaacaggttatcttcgtgcgcgaaaccccaaatacagccggggcaggggctactggcggcatcatcgggggtatcattgccgctatcattgccaccgccgtcgctgctacaggtattctgatatgtcgacagcaacggaaggaacagacactgcagggagccgaggaggatgaggaccttgagggccccccttcttacaagcctccaacacccaaggctaagctggaagcccaagagatgcctagccagctgtttactctcggcgccagcgagcactcacccctgaaaaccccttatttcgatgctggcgcatcctgcacagaacaagagatgccccggtaccacgaactgcctaccttggaggaaagatccggcccattgcatcctggcgcaacctctctcggcagtccaatccctgtacctcctggcccccctgcagtggaggatgtttccctggatctggaggacgaagagggcgaggaggaggaagagtatttggacaagatcaaccctatctacgacgccctcagctattcctctccatctgactcctaccagggtaaaggcttcgtcatgagccgcgctatgtatgtg

SEQ ID NO: 26 - DNA, 1614 bp, synthetic construct
  source 1..1614: mol_type = other DNA
atggctcgagctgtcgcacttctcccctccagatctcccccaacccccctgctgtggcctcttttgcttctgcttcttcgtaaaacaggcgctcaggacgttcgggtccaggtgctccccgaagtgagaggtcagctcggagggacagtggagcttccctgccacctccttccacctgttcccgggctgtatatctcccttgtgacctggcaaaggcctgatgcaccccctgaccaccaaaatgtggccgctttccacccaaaaatgggcccctcatttcccagcccaaagccaggaagccagagactgtccttcgtgagcgctaagcagtctaccaggcaggataccgaagctgagctgcaggatgctactcttgcattgcggggactgacagttgaggacgaagggaactacacatgcgagtttgcaacattcccaaaaggttccgtgaggggcatgacttggcttagggttattgctaaacctcagaatcacgctgaggcccaggaggtgactttttcccaagatcctgtgcccgtcgctaggtgtatctctaaggagggacggcccccagcccggatctcatggctttcaagtctcgattgggaggctaaggagactcaagtcagtgggacactcgccggaacagttaccgtaaccagccgattcacactcgttccaagcggacgcgcagatggggtcaccgtgacctgcaaggtcgaacatgaaagttttgaggaaccagccctgatacccgtgaccctgtcagttcggtacccacccgaggtgtccattagcggttacgacgataactggtaccttggccgcactgatgcaaccctgtcatgcgatgtgcactccaatccagaaccaactggatacgattggtccacaactagcggcatcttcccaacctctgccgtggctcaaggttcccagctcgtgatccacgccgttgacagtctgttcaacaccaccttcgtgtgtactgttactaacgccgtcggaatgggacgcgcagaacaggtcatctttgtgcgagaaacccctaacactgctggagctggggccacaggagggatcattggtggaattatcgcagccatcattgctacagccgtagccgctaccggcattctgatttgtagacagcagagaaaggaacaaacactgcagggtgccgaggaggacgaggatctggaggggcctccttcatacaaaccacctactccaaaggccaaattggaggagcaggagatgccctcacagttgtttaccttgggggcctccgaacactctcctttgaagacaccatacttcgacgccggggcttcctgtacagaacaggagatgccacggtaccatgagttgcccaccttggaagagaggtcaggacccctgcatcctggggctacctcactggggtctcctataccagtacctccaggccctccagtggtcgaggatgtaagtctggacttggaggacgaggaaggggaggaggaggaagagtacctggataaaattaatcccgtgtacgacgcactctcttactcctccccatccgacagctaccagggcaagggatttgtgatgtctagggcaatgtacgtg

(19) United States
(12) Patent Application Publication
(10) Pub. No.: US 2024/0376196 A1
(43) Pub. Date: Nov. 14, 2024

(54) CD112 ANTIBODY AND USE

(71) Applicant: HEFEI TG IMMUNOPHARMA CO., LTD., Hefei (CN)

(72) Inventors: Guoshuai CAO, Hefei, Anhui (CN); Ying CHENG, Hefei, Anhui (CN); Yangyang LI, Hefei, Anhui (CN); Yuwei WU, Hefei, Anhui (CN)

(73) Assignee: HEFEI TG IMMUNOPHARMA CO., LTD., Hefei (CN)

(21) Appl. No.: 18/657,532

(22) Filed: May 7, 2024

(30) Foreign Application Priority Data
May 9, 2023 (CN) CN 202310524678.9

(51) Int. Cl. C07K 16/28; A61K 39/00; A61P 35/00

(52) U.S. Cl. CPC C07K 16/2803; A61P 35/00; A61K 2039/505

(57) ABSTRACT

Provided are an antibody or an antigen-binding fragment thereof and use thereof. The antibody or antigen-binding fragment thereof includes light chain variable region CDRs and heavy chain variable region CDRs. A heavy chain variable region CDR1 sequence is as set forth in GFTFSSYX₁, where X₁ is Y or M. A heavy chain variable region CDR2 sequence is as set forth in INSQGGST. A heavy chain variable region CDR3 sequence is as set forth in ARSDYDWAWX₂AY, where X₂ is For Y. A light chain variable region CDR1 sequence is as set forth in QSLLYSSNQKNY. A light chain variable region CDR2 sequence is as set forth in WAX₃, where X₃ is S or N. A light chain variable region CDR3 sequence is as set forth in QQYYRYPPT.

### CROSS-REFERENCE TO RELATED APPLICATIONS

This application claims priority to Chinese Patent Application No. 202310524678.9, filed on May 9, 2023, the disclosure of which is hereby incorporated by reference in its entirety.

### STATEMENT REGARDING SEQUENCE LISTING

A Sequence Listing associated with this application is being filed concurrently herewith and is hereby incorporated by reference into the present specification. The file containing the Sequence listing is titled “Sequence_Listing. XML”, was created on Apr. 24, 2024, and is approximately 32,768 bytes in size.

### FIELD

The present disclosure relates to the field of antibodies. Particularly, the present disclosure relates to an antibody or an antigen-binding fragment thereof that can bind to CD112 and use thereof, more particularly, to an antibody or an antigen-binding fragment thereof, a nucleic acid molecule, an expression vector, a method for preparing the antibody or antigen-binding fragment thereof, a recombinant cell, a composition, and a use thereof, and a medicament and use thereof.

### BACKGROUND

Cancer is a major disease affecting survival and development of mankind. According to the latest data, the number of new cases of cancer is about 19 million, and the number of cancer-caused death cases is about 10 million every year worldwide. The incidence and mortality of cancers exhibit an increasing trend. In addition to surgical resection, traditional cancer treatment methods such as chemotherapy and radiotherapy have significant side effects and are prone to recurrence. In recent years, immunotherapy, including tumor-targeted antibodies, immune checkpoint antibodies, and bispecific antibodies, has become a new hot spot and hope for anti-cancer. Immunotherapeutic medicaments, including monoclonal antibodies and double antibodies, achieve anti-cancer effects mainly by promoting the function of autoimmune cells, with the characteristics of small side effects and long survival time of benefit patients.

In recent years, the immunotherapy represented by PD-1/L1 exhibits huge potential, and three types of immunotherapy, PD-1/L1, CTLA-4, and LAG-3, have been approved as medicaments. However, a non-negligible fact is in that more patients cannot benefit from the therapy even the total response ratio of the currently approved PD-1/L1 therapy with the most extensive indications is up to 30%.

An immune checkpoint molecule is an inhibitory molecule expressed on the surface of immune cells including T cells, NK cells, and mononuclear macrophages, capable of transmitting an inhibitory signal into the immune cells after binding to a corresponding ligand, thereby inhibiting the immune cells' functions against cancer. On the one hand, since immune checkpoint ligand expressions of tumors and tumor infiltrating lymphocytes have extremely high heterogeneity, the immune checkpoint therapy of single species is not universally suitable for all patients, and thus, most of the patients cannot benefit from the immune checkpoint therapy. On the other hand, some patients who have accepted the immune checkpoint therapy may have recurrent tumors and develop tolerance to the immune checkpoint therapy, such that continuous therapy may have no curative effects. For both reasons, it is necessary to develop immune checkpoint antibodies against more targets.

CD112 is a type I transmembrane protein that is highly expressed in a variety of tumor tissues. CD112 binds to the receptors such as CD112R, TIGIT, and CD226 on the surface of the immune cells, and transmits inhibitory or activating signals into immune cells through these receptors. To date, there are four kinds of CD112R Mabs worldwide (Compugen COM-701, Surface SRF-813, Junshi Biosciences JS-009, TG ImmunoPharma TGI-2), several TIGIT Mabs in the clinical stage, and one CD226 Mab (Eli Lily) in an early development stage. However, the development of medicaments for blocking CD112 antibody has not been disclosed worldwide, and CD112 antibody has different mechanisms of action, providing new hope for patients. Therefore, the development of medicaments for blocking CD112 antibody is of great significance for the treatment of tumors.

### SUMMARY

The present disclosure provides an antibody or an antigen-binding fragment thereof. The antibody or antigen-binding fragment thereof includes: light chain variable region CDRs and heavy chain variable region CDRs; wherein: a heavy chain variable region CDR1 sequence is as set forth in GFTFSSYX₁, where X₁ is Y or M; a heavy chain variable region CDR2 sequence is as set forth in INSQGGST (SEQ ID NO: 2); a heavy chain variable region CDR3 sequence is as set forth in ARSDYDWAWX₂AY, where X₂ is F or Y; a light chain variable region CDR1 sequence is as set forth in QSLLYSSNQKNY (SEQ ID NO: 4); a light chain variable region CDR2 sequence is as set forth in WAX₃, where X₃ is S or N; and a light chain variable region CDR3 sequence is as set forth in QQYYRYPPT (SEQ ID NO: 6).

The present disclosure provides a nucleic acid molecule. The nucleic acid molecule encodes the antibody or antigen-binding fragment thereof as described in the first aspect.

The present disclosure provides an expression vector. The expression vector carries the nucleic acid molecule of the second aspect.

The present disclosure provides a method for preparing the antibody or antigen-binding fragment thereof as described in the first aspect. The method includes: introducing an expression vector as described in the third aspect into cells; the expression vector carrying a nucleic acid molecule encoding the antibody or antigen-binding fragment thereof; and culturing the cells under conditions suitable for protein expression and secretion, to obtain the antibody or the antigen-binding fragment thereof; and culturing the cells under conditions suitable for protein expression and secretion, to obtain the antibody or antigen-binding fragment thereof, optionally, the cells are prokaryotic cells or eukaryotic cells; and optionally, the cells are eukaryotic cells.

The present disclosure provides a recombinant cell. The recombinant cell expresses the antibody or antigen-binding fragment thereof as described in the first aspect.

The present disclosure provides a composition. The composition includes the antibody or antigen-binding fragment thereof as described in the first aspect.

The present disclosure provides a method for preventing and/or treating a CD112-mediated disease. The method includes administrating the antibody or antigen-binding fragments thereof as described in the first aspect to a subject in need thereof.

The present disclosure provides a medicament. The medicament includes the antibody or antigen-binding fragment thereof as described in the first aspect, wherein the medicament is used for preventing and/or treating a CD112-mediated disease.

The present disclosure provides a kit. The kit includes the antibody or antigen-binding fragment thereof as described in the first aspect.

### BRIEF DESCRIPTION OF THE DRAWINGS

The foregoing and/or additional aspects and advantages of the present disclosure will become apparent and readily appreciated from the following description of the embodiments, taken in conjunction with the accompanying drawings, wherein:

FIG. 1 shows the ELISA result of the murine 9G11 antibody binding to the CD112 protein according to a specific embodiment of the present disclosure;

FIG. 2 shows the ELISA result of the murine 9G11 antibody blocking CD112 from binding to CD112R-Fc according to a specific embodiment of the present disclosure;

FIG. 3 shows the ELISA result of the murine 9G11 antibody blocking CD112 from binding to TIGIT according to a specific embodiment of the present disclosure;

FIG. 4 shows the ELISA result of the murine 9G11 antibody blocking CD112 from binding to CD226 according to a specific embodiment of the present disclosure;

FIG. 5A and FIG. 5B show the flow cytometry result of the chimeric antibody 9G11-hIgG4 and humanized antibody h9G11-hIgG4 binding to an A-375 malignant melanoma cell according to a specific embodiment of the present disclosure;

FIG. 6A and FIG. 6B show the flow cytometry result of chimeric antibody 9G11-hIgG4 and humanized antibody h9G11-hIgG4 binding to an A-549 non-small cell lung cancer according to a specific embodiment of the present disclosure;

FIG. 7 shows the affinity test result of humanized antibody h9G11-hIgG1 binding to the CD112 protein according to a specific embodiment of the present disclosure;

FIG. 8 shows the affinity detection result of the affinity mature antibody h9G11ZH21-hIgG1 binding to CD112 protein according to a specific embodiment of the present disclosure;

FIG. 9A and FIG. 9B show the flow cytometry result of humanized antibody h9G11-hIgG4 and affinity mature antibody h9G11ZH21-hIgG1 binding to an A-375 malignant melanoma cell according to a specific embodiment of the present disclosure;

FIG. 10A and FIG. 10B show the flow cytometry result of humanized antibody h9G11-hIgG4 and affinity mature antibody h9G11ZH21-hIgG1 binding to an MDA-MB-231 breast cancer cell according to a specific embodiment of the present disclosure;

FIG. 11A and FIG. 11B show the flow cytometry result of affinity mature antibody h9G11ZH21-hIgG1 binding to a CHO-K1-human CD112 cell according to a specific embodiment of the present disclosure;

FIG. 12A and FIG. 12B show the flow cytometry result of affinity mature antibody h9G11ZH21-hIgG1 binding to a CHO-K1-cynomolgus CD112 cell according to a specific embodiment of the present disclosure;

FIG. 13 shows the ELISA result of affinity mature antibody h9G11ZH21-hIgG1 blocking CD112 from binding to CD112R-Fc according to a specific embodiment of the present disclosure;

FIG. 14 shows the ELISA result of affinity mature antibody h9G11ZH21-hIgG1 blocking CD112 from binding to CD226 according to a specific embodiment of the present disclosure;

FIG. 15A and FIG. 15B show the flow cytometry result of affinity mature antibody h9G11ZH21-hIgG1 blocking CD112R from binding to a CHO-K1-human CD112 cell according to a specific embodiment of the present disclosure;

FIG. 16A and FIG. 11B show the flow cytometry result of affinity mature antibody h9G11ZH21-hIgG1 blocking CD226 from binding to a CHO-K1-human CD112 cell according to a specific embodiment of the present disclosure;

FIG. 17 shows the ELISA result of affinity mature antibody h9G11ZH21-hIgG1 promoting the secretion of IFN-γ by antigen-specific T cell according to a specific embodiment of the present disclosure;

FIG. 18 shows the ELISA result of affinity mature antibody h9G11ZH21-hIgG1 promoting the secretion of IL-2 by T cells in a mixed lymphocyte reaction according to a specific embodiment of the present disclosure; and

FIG. 19 shows the result of the affinity mature antibody h9G11ZH21-hIgG1 promoting the killing of the HepG2 liver cancer cells by NK92MI cells to according to a specific embodiment of the present disclosure.

### DETAILED DESCRIPTION

The present disclosure aims to solve, at least to some extent, at least one of the technical problems existing in the prior art. It is an object of the present disclosure to overcome the deficiencies of the prior art and to provide a CD112 antibody that can bind to CD112 and block CD112 from binding to its receptors CD112R, CD226, and TIGIT, and use thereof. The antibody provided by the present disclosure can promote the anti-cancer function of the immune cells.

Therefore, in a first aspect of the present disclosure, provided is an antibody or an antigen-binding fragment thereof. According to an embodiment of the present disclosure, the antibody or antigen-binding fragment thereof includes: light chain variable region CDRs and heavy chain variable region CDRs; wherein: a heavy chain variable region CDR1 sequence is as set forth in GFTFSSYX₁, where X₁ is Y or M; a heavy chain variable region CDR2 sequence is as set forth in INSQGGST (SEQ ID NO: 2); a heavy chain variable region CDR3 sequence is as set forth in ARSDYDWAWX₂AY, where X₂ is F or Y; a light chain variable region CDR1 sequence is as set forth in QSLLYSSNQKNY (SEQ ID NO: 4); a light chain variable region CDR2 sequence is as set forth in WAX₃, where X₃ is S or N; and a light chain variable region CDR3 sequence is as set forth in QQYYRYPPT (SEQ ID NO: 6). The present inventors have found through extensive experiments that the above-mentioned antibody or the antigen-binding fragment thereof can bind to human and monkey CD112, block CD112 from binding to its receptors CD112R, CD226, and TIGIT, and promote the anti-cancer function of immune cells.

In a second aspect of the present disclosure, provided is a nucleic acid molecule. According to an embodiment of the present disclosure, the nucleic acid molecule encodes the antibody or antigen-binding fragment thereof as described in the first aspect. The antibody or antigen-binding fragment thereof encoded by the first nucleic acid molecule according to an embodiment of the present disclosure can bind to human and monkey CD112, block CD112 from binding to CD112R, CD226, and TIGIT, and promote the anti-cancer function of immune cells.

In a third aspect of the present disclosure, provided is an expression vector. According to an embodiment of the present disclosure, the expression vector carries the nucleic acid molecule of the second aspect. The first expression vector according to an embodiment of the present disclosure, when introduced into a suitable recipient cell, is effective in achieving the binding of the antibody or antigen-binding fragment thereof as described in the first aspect to human and monkey CD112 and blocking CD112 from binding to its receptors, CD112R, CD226 and TIGIT under the mediation of the regulatory system.

In a fourth aspect of the present disclosure, provided is a method for preparing the antibody or antigen-binding fragment thereof as described in the first aspect. According to an embodiment of the present disclosure, the method includes: introducing the expression vector as described in the third aspect into cells; and culturing the cells under conditions suitable for protein expression and secretion, to obtain the antibody or the antigen-binding fragment thereof.

In a fifth aspect of the present disclosure, provided is a recombinant cell. According to an embodiment of the present disclosure, the recombinant cell expresses the antibody or antigen-binding fragment thereof as described in the first aspect, carries the nucleic acid molecule as described in the second aspect, or the expression vector as described in the third aspect.

In a sixth aspect of the present disclosure, provided is a composition. According to an embodiment of the present disclosure, the composition includes the antibody or antigen-binding fragment thereof as described in the first aspect, the nucleic acid molecule as described in the second aspect, the expression vector as described in the third aspect, or the recombinant cell as described in the fifth aspect.

In a seventh aspect of the present disclosure, provided is a use of the antibody or antigen-binding fragments thereof as described in the first aspect, the nucleic acid molecule as described in the second aspect, the expression vector as described in the third aspect, the recombinant cell as described in the fifth aspect, or the composition as described in the sixth aspect in the preparation of a medicament for the prevention and/or treatment of a CD112-mediated disease.

In an eighth aspect of the present disclosure, provided is a medicament. According to an embodiment of the present disclosure, the medicament includes the antibody or antigen-binding fragment thereof as described in the first aspect, the nucleic acid molecule as described in the second aspect, the expression vector as described in the third aspect, the recombinant cell as described in the fifth aspect, or the composition as described in the sixth aspect, wherein the medicament is used for preventing and/or treating a CD112-mediated disease.

In a ninth aspect of the present disclosure, provided is a use of the antibody or antigen-binding fragment thereof as described in the first aspect, the nucleic acid molecule as described in the second aspect, the expression vectors as described in the third aspect, or the recombinant cell as described in the fifth aspect in the preparation of a kit for detecting CD112.

In a tenth aspect of the present disclosure, provided is a kit. According to an embodiment of the present disclosure, the kit includes the antibody or antigen-binding fragment thereof as described in the first aspect, the nucleic acid molecule as described in the second aspect, the expression vector as described in the third aspect, or the recombinant cell as described in the fifth aspect.

In an eleventh aspect of the present disclosure, provided is a method for preventing and/or treating a CD112-mediated related disease. According to an embodiment of the present disclosure, the method includes administering to a subject at least one of: 1) the antibody or antigen-binding fragment thereof as described in the first aspect; 2) the nucleic acid molecule as described in the second aspect; 3) the expression vector as described in the third aspect; 4) the recombinant cell as described in the fifth aspect; 5) the composition described in the sixth aspect; and 6) the medicament described in the eighth aspect. As previously described, the antibody or antigen-binding fragment can bind to human and monkey CD112 protein and can effectively treat or prevent a CD112-mediated related disease, and thus, the method according to an embodiment of the present disclosure can effectively treat or prevent a CD112-mediated related disease.

In a twelfth aspect of the present disclosure, provided is a method for diagnosing a CD112-mediated related disease. According to an embodiment of the present disclosure, the method includes using at least one of the following to detect the CD112 in a sample to be tested: 1) the antibody or antigen-binding fragment thereof as described in the first aspect; 2) the nucleic acid molecule as described in the second aspect; 3) the expression vector described as described in the third aspect; and 4) the recombinant cell as described in the fifth aspect; determining the content of CD112 in the sample to be tested based on the detection result of CD112. The antibody or antigen-binding fragment, or the antibody or antigen-binding fragment expressed by the nucleic acid molecule, the expression vector, or the recombinant cell, as provided in the present disclosure, can effectively bind to the human and monkey CD112 protein, or the antibody or antigen-binding fragment expressed by the nucleic acid molecule, the expression vector, and the recombinant cell can effectively bind to CD112. Therefore, the method described in present disclosure can effectively detect the content of CD112 in a sample to be tested from a subject, and can effectively diagnose a related disease caused by CD112.

In a thirteenth aspect of the present disclosure, provided is a method for staging a CD112-mediated related disease. According to an embodiment of the present disclosure, the method includes using at least one of the following to detect the CD112 in a sample to be tested: 1) the antibody or antigen-binding fragment thereof as described in the first aspect; 2) the nucleic acid molecule as described in the second aspect; 3) the expression vector as described in the third aspect; and 4) the recombinant cell as described in the fifth aspect; determining the content of CD112 in the sample to be tested based on the detection result of CD11. The antibody or antigen-binding fragment, or the antibody or antigen-binding fragment expressed by the nucleic acid molecule, the expression vector, and the recombinant cell, as provided in the present disclosure, can effectively bind to human and monkey CD112. Therefore, the method described in the present disclosure can effectively detect the content of CD112 in a sample to be tested from a subject, and evaluating the stage of a related disease caused by CD112 based on the content of CD112.

In a fourteenth aspect of the present disclosure, provided is a method for evaluating the prognosis of a CD112-mediated related disease. According to an embodiment of the present disclosure, the method includes using at least one of the following to detect the CD112 in a sample to be tested: 1) the antibody or antigen-binding fragment thereof as described in the first aspect; 2) the nucleic acid molecule as described in the second aspect; 3) the expression vector as described in the third aspect; and 4) the recombinant cell as described in the fifth aspect; determining the content of CD112 in the sample to be tested based on the detection result of CD112. As previously described, the content of CD112 has an important effect on cancer. After the treatment of an individual with the related disease, the prognosis of such a disease can be effectively evaluated by monitoring the content of CD112 in a tissue or excretion, such as peripheral blood, urine, etc., for example, by comparing the content of CD112 in a subject before and after treatment, or comparing the content of CD112 in a subject after treatment with the level of CD112 in a normal individual or an individual with a disease, etc. The antibody or antigen-binding fragment, or the antibody or antigen-binding fragment expressed by the nucleic acid molecule, the expression vector, and the recombinant cell, as provided in the present disclosure, can effectively bind to human and monkey CD112. Therefore, the method described in the present disclosure can effectively detect the content of CD112 in a sample to be tested from a subject, and evaluate the prognosis of a related disease caused by CD112 based on the content of CD112.

In a fifteenth aspect of the present disclosure, provided is a use of the antibody or the antigen-binding fragment thereof as described in the first aspect, the nucleic acid molecule as described in the second aspect, the expression vector as described in the third aspect, the recombinant cell as described in the fifth aspect, the composition as described in the sixth aspect, or the medicament as described in the eighth aspect in treating or preventing a CD112-mediated related disease. As previously described, the antibody or antigen-binding fragment can effectively bind to human and monkey CD112 and can effectively treat or prevent a CD112-mediated related disease.

In a sixteenth aspect of the present disclosure, provided is a use of the antibody or antigen-binding fragment as described in the first aspect, the nucleic acid molecule as described in the second aspect, the expression vector as described in the third aspect, or the recombinant cell as described in the fifth aspect in diagnosing or staging a CD112-mediated related disease, or evaluating prognosis of a CD112-mediated related disease. As previously described, the antibody or antigen-binding fragment, or the antibody or antigen-binding fragment expressed by the nucleic acid molecule, expression vector, or recombinant cell provided in the present application can effectively bind to human and monkey CD112. Therefore, the method described in the present application can effectively detect the content of CD112 in a sample to be tested from a subject, and can effectively diagnose, stage and evaluate the prognosis of the related diseases mediated by CD112.

Additional aspects and advantages of the present disclosure will be set forth in part in the description which follows and, in part, will be obvious from the description, or may be learned by practice of the present disclosure.

Below, the embodiments of the present disclosure will be described in more detail. The embodiments described below are exemplary and are intended to be illustrative of the present disclosure only and are not to be construed as limiting the present disclosure.

In order to facilitate understanding of the present disclosure, some technical and scientific terms are specifically defined below. Unless clearly defined elsewhere in the present document, all other technical and scientific terms used in the present document have the meanings commonly understood by those skilled in the art to which the present disclosure belongs. An abbreviation of amino acid residues is a standard 3-letter and/or 1-letter code that refers to one of the 20 commonly used L-amine acids in the art.

It should be noted that the terms “first” and “second” are used for descriptive purposes only and are not to be construed as indicating or implying relative importance or implicitly indicating the number of technical features indicated. Thus, a feature defined as “first” or “second” may explicitly or implicitly includes one or more of these features. Furthermore, in the description of the present disclosure, unless otherwise specified, “multiple” means two or more.

The terms “comprising” or “including” herein are used in an open-ended fashion, i.e. to include what is specified in the present disclosure, but not to exclude other aspects.

The terms “optionally”, “optional” or “option” herein usually refers to that the subsequent events or conditions that may but may not necessarily occur, and the description includes the circumstances in which the event or condition occurs, as well as the circumstances in which the event or condition does not occur.

As used herein, the term “antibody” generally refers to an immunoglobulin molecule including two light chains with lighter molecular weight and two heavy chains with heavier molecular weight, the heavy (H) and light (L) chains being joined by disulfide bonds to form a tetrapeptide chain molecule. Among them, the amino-terminal (N-terminal) amino acid sequence of peptide chain varies greatly and is called variable region (V-region), and the carboxy-terminal (C-terminal) is relatively stable and varies little and is called constant region (C-region). The V regions of the L chain and H chain are referred to as VL and VH, respectively. Certain regions in the variable region have a higher degree of variation in amino acid composition and arrangement, referred to as a hypervariable region (HVR), which is the location where the antigen and the antibody bind and thus also referred to as a complementarity-determining region (CDR). There are three CDRs on both the heavy chain variable region and light chain variable region.

In the present disclosure, the term antigen-binding fragment, i.e. “antibody fragment” used, when not stated to the contrary, generally refers to an antigen-binding antibody fragment and may include a portion of a complete antibody, typically an antigen-binding region or a variable region. Examples of antibody fragments include Fab, Fab′, F(ab′) 2, Fv or scFv, diabody, linear antibody, single-chain antibody molecules, etc.

The term “complementarity determining region”, “CDR” or “CDR sequence” refers to the amino acid sequence of an antibody that is responsible for antigen-binding, and for example, it generally includes amino acid residues near 23-34 (L1), 50-56 (L2) and 89-97 (L3) in a light chain variable region, and amino acid residues near 31-35B (H1), 50-65 (H2) and 95-102 (H3) in a heavy chain variable region (Kabat et al. Sequences of Proteins of Immunological Interest, 5th Ed. Public Health Service, National Institutes of Health, Bethesda, MD. (1991)); and/or amino acid residues from the “hypervariable loop” (for instance, amino acid residues near 26-32 (LI), 50-52 (L2), and 91-96 (L3) in the light chain variable region, and 26-32 (H1), 53-55 (H2), and 96-101 (H3) in the heavy chain variable region (Chothia and Lesk J.Mol.Biol. 196:901-917 (1987)).

As used herein, the term “frame region” or “framework region”, abbreviated FR, refers to the non-CDR portion of the variable region of the heavy or light chain of an antibody. Among them, there are about 110 amino acid sequences near N-terminus of the H and L chains of the antibody with significant changes, while the amino acid sequences of other parts are relatively constant. Based on this, the light chain and the heavy chain can be divided into a variable region (V region) and a constant region (C region), wherein the variable region includes a hypervariable region (HVR) or a complementary determining region (CDR) and a frame region. FR has lower variability than CDR. There are four FR molecules, FR₁, FR₂, FR₃, and FR₄. Upon recognition of the antibody, the curling of the four FR molecules causes the CDR molecules to approach each other.

Those skilled in the art may substitute, add, and/or delete one or more (e.g. 1, 2, 3, 4, 5, 6, 7, 8, 9, or 10 or more) amino acids to a sequence of the present disclosure to obtain a variant of the sequence of the antibody or functional fragment thereof without substantially affecting the activity of the antibody (retaining at least 95% activity). They are considered to be included within the scope of the present disclosure. For example, amino acids having similar properties may be substituted in the variable region. The sequence of the variant of the present disclosure may have at least 80%, 81%, 82%, 83%, 84%, 85%, 86%, 87%, 88%, 89%, 90%, 91%, 92%, 93%, 94%, 95%, 96%, 97%, 98%, 99%, 99.5% or 99.9% identity (or homology) to the reference sequence. Sequence identity described herein can be measured using sequence analysis software, e.g. a computer program BLAST using default parameters, in particular BLASTP or TBLASTN. Amino acid sequences referred in the present disclosure are set forth from the N-terminus to the C-terminus.

As previously described, the antibody of the present disclosure may be full length antibody (e.g. IgG1 or IgG4 antibodies) or may include only an antigen-binding portions (e.g. Fab, F(ab′) 2 or scFv fragment), or may be modified to affect function. The present disclosure includes an anti-CD112 antibody having modified glycosylation patterns. In some applications, it may be useful to make modifications to remove undesired glycosylation sites, or to eliminate the presence of fucose moieties on the oligosaccharide chains, e.g. to enhance antibody-dependent cellular cytotoxicity (ADCC) function. In other applications, galactosylation modifications can be made to alter complement-dependent cellular cytotoxicity (CDC).

The term “functional fragment” as used herein refers in particular to an antibody fragment such as Fv, scFv (sc means single chain), Fab, F(ab′) 2, Fab′, scFv-Fc fragment or diabody, or any fragment which should be capable of increasing the half-life by chemical modification, e.g. addition of polyalkylene glycols, such as polyethylene glycol (“PEGylation, PEG”) (referred to as pegylated fragments of Fv-PEG, scFv-PEG, Fab-PEG, F(ab′) 2-PEG or Fab′-PEG) (“PEG” means polyethylene glycol), these fragments having CD112 binding activity. Preferably, the functional fragment will consist of or contain partial sequences of the heavy chain variable region or light chain variable region of its source antibody, sufficient to retain the same binding specificity and sufficient affinity as its source antibody. For CD112, it is preferred to be at least 1/100 of its source antibody affinity, and in a more preferred manner, at least 1/10. Such functional fragments will include a minimum of 5 amino acids, preferably 10, 15, 25, 50, and 100 contiguous amino acids of the antibody sequence from which they are derived.

As used herein, the terms “identity”, “homology”, or “similarity”, when used to describe an amino acid sequence or a nucleic acid sequence relative to a reference sequence, refer to the percentage of the same amino acids or nucleotides between two amino acid sequences or nucleic acid sequences is determined by conventional methods, for instance, referring to Ausubel et al. eds. (1995), Current Protocols in Molecular Biology, chapter 19 (Greene Publishing and Wiley-Interscience, New York); and the ALIGN program (Dayhoff (1978), Atlas of Protein Sequence and Structure 5: Suppl.3 (National Biomedical Research Foundation, Washington, D.C.). Various algorithms are applicable to sequence alignment and sequence identity determination, including, a homology alignment algorithm of Needleman et al., (1970) J. Mol. Biol. 48:443; a local homology algorithm of Smith et al., (1981) Adv. Appl. Math. 2:482; a similarity search method of Pearson et al., (1988) Proc. Natl. Acad. Sci. 85:2444; a Smith-Waterman algorithm (Meth. Mol. Biol. 70:173-187 (1997); and the BLASTP, BLASTN, and BLASTX algorithms (see Altschul et al., (1990) J. Mol. Biol. 215:403-410). Computer programs utilizing these algorithms are also available and include, but are not limited to: ALIGN or Megalign (DNASTAR) software, or WU-BLAST-2 (Altschul et al. Meth. Enzym., 266:460-480 (1996)); or GAP, BESTFIT, BLAST Altschul et al., supra, FASTA, and TFASTA, available in the Genetics Computing Group (GCG) package, 8th edition, Madison, Wisconsin, USA; and CLUSTAL in the PC/Gene program provided by Intelligenetics, Mountain View, California.

As used herein, the term “nucleotide” generally refers to a ribonucleotide, a deoxynucleotide, or a modified form of any type of nucleotide, and a combination thereof.

As used herein, the term “at least 80% similarity” means at least 80%, and may be 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9% similarity to each reference sequence.

As used herein, the term “expression vector” generally refers to a nucleic acid molecule that can be inserted into a suitable host for self-replication, which transfers the inserted nucleic acid molecule into and/or between host cells. The expression vector may include a vector for mainly inserting DNA or RNA into a cell, a vector for mainly replicating DNA or RNA, and an expression vector for mainly transcripting and/or translating of DNA or RNA. The expression vector also includes a vector having a variety of the above functions. The expression vector may be a polynucleotide capable of being transcribed and translated into a polypeptide when introduced into a suitable host cell. Typically, the expression vector may produce the desired expression product by culturing a suitable host cell containing the expression vector.

As used herein, the term “recombinant cell” generally refers to a cell with unique characteristics of stable inheritance obtained by modifying or recombining the genetic material of host cells by genetic engineering technology or cell fusion technology. The term “host cell” refers to prokaryotic or eukaryotic cells that can be introduced into a recombinant expression vector. As used herein, the term “transformed” or “transfected” refers to the introduction of a nucleic acid (e.g. a vector) into a cell by various techniques known in the art. Suitable host cells may be transformed or transfected with the DNA sequences of the present disclosure and may be used for the expression and/or secretion of a target protein. Examples of suitable host cells in the present disclosure include immortalized hybridoma cells, NS/0 myeloma cells, 293 cells, Chinese hamster ovary (CHO) cells, HeLa cells, Cap cells (human amniotic fluid-derived cells), insect cells, PER.C6 cells, and CoS cells.

As used herein, the term “fusion protein” refers to a novel protein formed by the fusion of at least two proteins or polypeptides, which can generally be achieved by techniques such as genetic engineering, e.g. the expression product of two genes obtained by DNA recombination technology.

As used herein, the term “pharmaceutical composition” generally refers to a unit dosage form and may be prepared by any of the methods well known in the pharmaceutical art. All methods include the step of combining the active ingredient with a carrier that constitutes one or more accessory ingredients. In general, the compositions are prepared by uniformly and sufficiently combining the active compound with a liquid carrier, a finely divided solid carrier, or both.

The term “pharmaceutically acceptable” herein refers to that the substance or composition must be compatible chemically and/or toxicologically, with the other ingredients contained in the formulation, and/or the mammal being treated therewith. Preferably, “pharmaceutically acceptable” of the present disclosure refers to those approved by federal regulatory agencies or national governments, or listed in the United States Pharmacopoeia or other generally recognized pharmacopoeia for use in animals, especially in humans.

As used herein, the term “pharmaceutically acceptable excipient” or “pharmaceutically acceptable carrier” can include any solvent, solid excipient, diluent, or other liquid excipient, etc. suitable for the particular desired dosage form. Except insofar as any conventional adjuvant is incompatible with the compounds of the present disclosure, such as by producing any undesirable biological effect or interacting in a deleterious manner with any other component(s) of the pharmaceutically acceptable composition, its use is contemplated to be within the scope of this invention.

For other pharmaceutically acceptable excipients or carriers mentioned herein, as well as their processes, literature on this topic can be referred to, in particular to Handbook of Pharmaceutical Excipients, 3rd edition, Arthur H. Kibbe, ed. American Pharmaceutical Association, Washington, USA and Pharmaceutical Press, London; and Lexikon der Hilfsstoffe für Pharmazie, Kosmetik and angrenzende Gebiete, H. P. Fiedler, ed., 4th edition, Cantor, Aulendorf, ed., and earlier editions.

As used herein, the term “administering” refers to introducing a predetermined amount of a substance into a patient in some suitable manner. The fusion protein or pharmaceutical composition of the present disclosure may be administered by any convenient route, so long as it reaches the desired tissue. Various modes of administration are contemplated, including peritoneal, intravenous, intramuscular, subcutaneous, etc. but the present disclosure is not limited to these exemplary modes of administration. Preferably, the compositions of the present disclosure are administered by intravenous or subcutaneous injection.

As used herein, the term “treatment” refers to obtaining a desired pharmacologic and/or physiologic effect. The effect may be preventive in terms of completely or partially preventing a disease or symptom thereof and/or may be therapeutic in terms of partially or completely curing a disease and/or adverse effect caused by the disease. As used herein, “treatment” encompasses diseases of mammals, particularly humans, including (a) preventing the occurrence of the disease or condition in an individual who is predisposed to the disease but has not yet been diagnosed; (b) inhibiting the disease, for example, arresting the development of the disease; or (c) relieving the disease, e.g. alleviating symptoms associated with the disease. As used herein, “treatment” encompasses any administration of a medicament or compound to a subject to treat, cure, alleviate, ameliorate, lessen, or inhibit disease in the subject, including, but not limited to, administration of a medicament including a compound described herein to a subject in need thereof.

The present disclosure provides an antibody or an antigen-binding fragment, nucleic acid molecule, expression vector, recombinant cell, composition, medicament, and use thereof, which will be described in detail below.

### Antibody or Antigen-Binding Fragment Thereof

The present disclosure provides an antibody or antigen-binding fragment thereof that can bind to CD112, wherein the antibody includes heavy chain CDR1, heavy chain CDR2, and heavy chain CDR3; and light chain CDR1, light chain CDR2, and light chain CDR3. The antibody or antigen-binding fragment thereof can bind to human and monkey CD112, block CD112 from binding to CD112R, CD226, and TIGIT, and promote the anti-cancer function of immune cells.

According to the present disclosure, the antibody can bind to both human and monkey CD112, particularly the amino acid sequence set forth in SEQ ID NO: 25.

(SEQ ID NO: 25)
MARAAALLPSRSPPTPLLWPLLLLLLLETGAQDVRVQVLPEVRGQ
LGGTVELPCHLLPPVPGLYISLVTWQRPDAPANHQNVAAFHPKMG
PSFPSPKPGSERLSFVSAKQSTGQDTEAELQDATLALHGLTVEDE
GNYTCEFATFPKGSVRGMTWLRVIAKPKNQAEAQKVTFSQDPTTV
ALCISKEGRPPARISWLSSLDWEAKETQVSGTLAGTVTVTSRFTL
VPSGRADGVTVTCKVEHESFEEPALIPVTLSVRYPPEVSISGYDD
NWYLGRTDATLSCDVRSNPEPTGYDWSTTSGTFPTSAVAQGSQLV
IHAVDSLFNTTFVCTVTNAVGMGRAEQVIFVRETPNTAGAGATGG
IIGGIIAAIIATAVAATGILICRQQRKEQTLQGAEEDEDLEGPPS
YKPPTPKAKLEAQEMPSQLFTLGASEHSPLKTPYFDAGASCTEQE
MPRYHELPTLEERSGPLHPGATSLGSPIPVPPGPPAVEDVSLDLE
DEEGEEEEEYLDKINPIYDALSYSSPSDSYQGKGFVMSRAMYV.

In a preferred embodiment of the present disclosure, in order to further increase the biological acceptability of the antibody, the antibody may also be humanized, that is, the antibody is a chimeric antibody or humanized antibody. The term “chimeric antibody” refers to a recombinant antibody obtained by substituting the amino acid sequence of the constant region of a monoclonal antibody from one species (such as mouse) with the constant region of an antibody from another species (such as human) by utilizing recombinant DNA technology. The term “humanized antibody” refers to a recombinant antibody obtained by completely substituting the constant region and the amino acid sequences of the non-CDR (Fv frame region (FR)) of the variable region of a monoclonal antibody from one species (such as mouse) with the the constant region and the amino acid sequences of non-CDR of an antibody from another species (e.g. human) by utilizing the recombinant DNA technology. That is, when the constant region of an antibody is humanized, the antibody is referred to as a chimeric antibody, and after all of the constant region and the non-CDR amino acid sequences of the variable regions are humanized, the antibody is referred to as a humanized antibody. The humanized method may be conducted by referring to a conventional antibody engineering technology, which is not described in detail herein. The sequence of the heavy chain variable region of the humanized antibody provided by the present disclosure is as set forth in SEQ ID NO: 12, and the sequence of the light chain variable region is as set forth in SEQ ID NO: 13.

(SEQ ID NO: 12)
EVQLVESGGGLVQPGGSLRLSCAASGFTFSSYYMSWVRQAPGKRL
ELVAAINSQGGSTYYPDTVKGRFTISRDNAKNTLYLQMNSLRAED
TAVYYCARSDYDWAWFAYWGQGTLVTVSS.
(SEQ ID NO: 13)
DIVMTQSPSSLAVSLGERATINCKSSQSLLYSSNQKNYLAWYQQK
PGQPPKLLIYWASTRESGVPDRFSGSGSGTDFTLTISSLQAEDVA
VYYCQQYYRYPPTFGGGTKLEIK.

Therefore, in a first aspect of the present disclosure, provided is an antibody or an antigen-binding fragment thereof. According to an embodiment of the present disclosure, the antibody or antigen-binding fragment thereof includes: light chain variable region CDRs and heavy chain variable region CDRs; wherein a heavy chain variable region CDR1 sequence is as set forth in GFTFSSYX₁; a heavy chain variable region CDR2 sequence is as set forth in INSQGGST (SEQ ID NO: 2); a heavy chain variable region CDR3 sequence is as set forth in ARSDYDWAWX₂AY; X₁ is Y or M, and X₂ is F or Y; a light chain variable region CDR1 sequence is as set forth in QSLLYSSNQKNY (SEQ ID NO: 4); a light chain variable region CDR2 sequence is as set forth in WAX₃; a light chain variable region CDR3 sequence is as set forth in QQYYRYPPT (SEQ ID NO: 6); X₃ is S or N.

According to an embodiment of the present disclosure, the antibody is selected from at least one of the following CDR sequences: heavy chain variable region CDR sequence: SEQ ID NO: 1, SEQ ID NO: 2, SEQ ID NO: 3, SEQ ID NO: 7, and SEQ ID NO: 8; light chain variable region CDR sequences: SEQ ID NO: 4, SEQ ID NO: 5, SEQ ID NO: 6, and SEQ ID NO: 9.

(SEQ ID NO: 1)
GFTFSSYY.
(SEQ ID NO: 2)
INSQGGST.
(SEQ ID NO: 3)
ARSDYDWAWFAY.
(SEQ ID NO: 4)
QSLLYSSNQKNY.
(SEQ ID NO: 5)
WAS.
(SEQ ID NO: 6)
QQYYRYPPT.
(SEQ ID NO: 7)
GFTFSSYM.
(SEQ ID NO: 8)
ARSDYDWAWYAY.
(SEQ ID NO: 9)
WAN.

According to an embodiment of the present disclosure, the heavy chain variable region CDR sequence has at least 80% similarity to any one of the sequences as set forth in SEQ ID NO: 1 to SEQ ID NO: 3 or SEQ ID NO: 7 to SEQ ID NO:8; the light chain variable region CDR sequence has at least 80% similarity to any one of the sequences as set forth in SEQ ID NO: 4 to SEQ ID NO: 6, or SEQ ID NO: 9.

According to an embodiment of the present disclosure, the antibody or antigen-binding fragment thereof includes: heavy chain variable region CDR1 sequence, heavy chain variable region CDR2 sequence, and heavy chain variable region CDR3 sequence as set forth in SEQ ID NO: 1, SEQ ID NO: 2, and SEQ ID NO: 3, respectively, or as set forth in amino acid sequences having at least 80% similarity to SEQ ID NO: 1, SEQ ID NO: 2, and SEQ ID NO: 3, respectively; or the heavy chain variable region CDR1 sequence, the heavy chain variable region CDR2 sequence, he heavy chain variable region CDR3 sequence as set forth in SEQ ID NO: 7, SEQ ID NO: 2, and SEQ ID NO: 8, respectively, or as set forth in amino acid sequences having at least 80% similarity to SEQ ID NO: 7, SEQ ID NO: 2, and SEQ ID NO: 8, respectively.

According to an embodiment of the present disclosure, the antibody or antigen-binding fragment thereof includes the light chain variable region CDR1 sequence, the light chain variable region CDR2 sequence, the light chain variable region CDR3 sequence as set forth in SEQ ID NO: 4, SEQ ID NO: 5, and SEQ ID NO: 6, respectively, or as set forth in amino acid sequences having at least 80% similarity to SEQ ID NO: 4, SEQ ID NO: 5, and SEQ ID NO: 6, respectively; or the light chain variable region CDR1 sequence, the light chain variable region CDR2 sequence, the light chain variable region CDR3 sequence as set forth in SEQ ID NO: 4, SEQ ID NO: 9, and SEQ ID NO: 6, respectively, or as set forth in amino acid sequences having at least 80% similarity to SEQ ID NO: 4, SEQ ID NO: 9, and SEQ ID NO: 6, respectively.

According to an embodiment of the present disclosure, the antibody or antigen-binding fragment thereof includes at least one of a heavy chain frame region sequence and a light chain frame region sequence; wherein at least one part of at least one of the heavy chain frame region sequence and the light chain frame region sequence is derived from at least one of a murine antibody, a humanized antibody, a primate-derived antibody, or a mutant thereof.

According to an embodiment of the present disclosure, the antibody or antigen-binding fragment thereof has a heavy chain variable region having an amino acid sequence as set forth in SEQ ID NO: 10, SEQ ID NO: 12, or SEQ ID NO: 14; and/or the antibody or the antigen-binding fragment thereof has a light chain variable region with an amino acid sequence as set forth in SEQ ID NO: 11, SEQ ID NO: 13, or SEQ ID NO: 15.

(SEQ ID NO: 10)
DVKLVESGGGLVKLGGSLKLSCAASGFTFSSYYMSWVRQTPEKRL
ELVAAINSQGGSTYYPDTVKGRFTISRDNAKNTLYLQMSSLKSED
TGLYYCARSDYDWAWFAYWGQGTLVTVSA.
(SEQ ID NO: 11)
DIVMSQSPSSLAVSVGEKVAMSCKSSQSLLYSSNQKNYLAWYQQK
PGQSPKLLIYWASTRESGVPDRFTGSGSGTDFTLTISSVKAEDLA
VYYCQQYYRYPPTFGAGTKLELK.
(SEQ ID NO: 12)
EVQLVESGGGLVQPGGSLRLSCAASGFTFSSYYMSWVRQAPGKRL
ELVAAINSQGGSTYYPDTVKGRFTISRDNAKNTLYLQMNSLRAED
TAVYYCARSDYDWAWFAYWGQGTLVTVSS.
(SEQ ID NO: 13)
DIVMTQSPSSLAVSLGERATINCKSSQSLLYSSNQKNYLAWYQQK
PGQPPKLLIYWASTRESGVPDRFSGSGSGTDFTLTISSLQAEDV
AVYYCQQYYRYPPTFGGGTKLEIK.
(SEQ ID NO: 14)
EVQLVESGGGLVQPGGSLRLSCAASGFTFSSYMMSWVRQAPGKRL
ELVAAINSQGGSTYYPDTVEGRFTISRDNAKNTLYLQMNSLRAED
TAVYYCARSDYDWAWYAYWGQGTLVTVSS.
(SEQ ID NO: 15)
DIVMTQSPSSLAVSLGERATINCKSSQSLLYSSNQKNYLAWYQQK
PGQPPKLLIYWANTRESGVPDRFSGSGSGTDFTLTISSLQAEDVA
VYYCQQYYRYPPTFGGGTKLEIK.

According to an embodiment of the present disclosure, the antibody or antigen-binding fragment thereof has a heavy chain variable region of an amino acid sequence as set forth in SEQ ID NO: 10, and a light chain variable region of an amino acid sequence as set forth in SEQ ID NO: 11, namely a murine heavy chain variable region and a light chain variable region.

According to an embodiment of the present disclosure, the antibody or antigen-binding fragment thereof has a heavy chain variable region of an amino acid sequence as set forth in SEQ ID NO: 12, and a light chain variable region of an amino acid sequence as set forth in SEQ ID NO: 13, namely a humanized heavy chain variable region and a light chain variable region.

According to an embodiment of the present disclosure, the antibody or antigen-binding fragment thereof includes a heavy chain variable region having an amino acid sequence as set forth in SEQ ID NO: 14, and a light chain variable region having an amino acid sequence as set forth in SEQ ID NO: 15, namely an affinity mature heavy chain variable region and a light chain variable region.

According to an embodiment of the present disclosure, the antibody or the antigen-binding fragment thereof includes at least one of a heavy chain constant region and light chain constant region, and at least one part of at least one of the heavy chain constant region and light chain constant region is derived from at least one of a humanized antibody, a primate-derived antibody, a mouse antibody, or a mutant thereof.

According to an embodiment of the present disclosure, the light chain constant region and the heavy chain constant region are both derived from a murine IgG1 antibody, a murine IgG 2a antibody, or a mutant thereof; or a humanized IgG1 antibody, a humanized IgG2 antibody, a humanized IgG3 antibody, a humanized IgG4 antibody, or a mutant thereof.

### Nucleic Acid Molecule, Expression Vector, and Recombinant Cell

In preparing or obtaining such antibodies or antigen-binding fragments thereof, nucleic acid molecules that express such antibodies or antigen-binding fragments thereof can be used to linked to different vectors and then expressed in different cells to obtain the corresponding antibodies.

For this, in a second aspect of the present disclosure, provided is a nucleic acid molecule. According to an embodiment of the present disclosure, the nucleic acid molecule encodes the antibody or antigen-binding fragment thereof according to the first aspect.

It will be appreciated by those skilled in the art that the features and advantages described above for the antibody or antigen-binding fragment thereof are equally applicable to the nucleic acid molecule and will not be described further herein.

In a third aspect of the present disclosure, provided is an expression vector. According to an embodiment of the present disclosure, the expression vector carries the nucleic acid molecule of the second aspect. When the isolated polynucleotide described above is linked to a first expression vector, the polynucleotide may be linked directly or indirectly to control elements on the first expression vector, so long as these control elements are capable of controlling the translation, expression, etc. of the polynucleotide. These control elements may of course be derived directly from the expression vector itself or may be exogenous, i.e. not derived from the expression vector itself. Of course, it is sufficient that the polynucleotide is operably linked to a control element.

The expression vectors provided in the examples of the present disclosure can efficiently express the aforementioned antibody or antigen-binding fragment thereof in a suitable recipient cell, and the antibody or antigen-binding fragment thereof designed in the present disclosure has stronger specificity and higher safety.

As used herein, “operably linked” means that the foreign gene is linked to a vector such that control elements within the vector, such as a transcription control sequence and a translation control sequence, etc. are capable of performing their intended function of regulating the transcription and translation of the foreign gene. Of course, the polynucleotides used to encode the heavy and light chains of the antibody may be separately inserted into different vectors, typically the same vector. Commonly used vectors may be, for example, plasmids, bacteriophages, and the like.

It will be appreciated by those skilled in the art that the features and advantages described above for the antibody or antigen-binding fragment thereof are equally applicable to the expression vector and will not be described further herein.

In a fifth aspect of the present disclosure, provided is a recombinant cell. According to an embodiment of the present disclosure, the recombinant cell expresses the antibody or antigen-binding fragment thereof of the first aspect, and carries the nucleic acid molecule of the second aspect, or the expression vector of the third aspect. The recombinant cell is obtained by transfecting or transforming the expression vector. According to some embodiments of the present disclosure, the recombinant cell can express the aforementioned antibody or antigen-binding fragment efficiently and massively under suitable conditions.

According to some embodiments of the present disclosure, the recombinant cell can express the antibody or antigen-binding fragment thereof efficiently and massively under suitable conditions, and the antibody or antigen-binding fragment thereof has stronger specificity, longer half-life, and higher efficacy, and can deliver the antibody medicament to the target cells at a lower dosage, realizing the effective treatment or prevention of a CD112-mediated disease with low toxic side effects and higher safety.

It is to be noted that the recombinant cell of the present disclosure is not particularly limited and may be a prokaryotic cell, a eukaryotic cell or a bacteriophage. The prokaryotic cell may be E. coli, Bacillus subtilis, Streptomyces or Proteus mirabilis, etc. The eukaryotic cells include fungus such as Pichia pastoris, Saccharomyces cerevisiae, Schizosaccharomyces pombe, and Trichoderma, insect cells such as Mythimna separata, plant cells such as tobacco, mammalian cells such as BHK cells, CHO cells, COS cells, myeloma cells, and the like. In some embodiments, the recombinant cells of the present disclosure are preferably mammalian cells, including BHK cells, CHO cells, NSO cells, or COS cells, and do not include animal germ cells, fertilized eggs, or embryonic stem cells.

It should be noted that “suitable conditions” as used in the description of the present application refers to conditions suitable for the expression of the antibody or antigen-binding fragment thereof described herein. Those skilled in the art will readily appreciate that conditions suitable for expression of the antibody or antigen-binding fragment include, but are not limited to, a suitable transformation or transfection mode, suitable transformation or transfection conditions, a healthy host cell state, a suitable host cell density, a suitable cell culture environment, and a suitable cell culture time. “Suitable conditions” are not particularly limited, and those skilled in the art can optimize the most suitable conditions for expression of the antibody or antigen-binding fragment according to the particular circumstances of the laboratory.

It will be appreciated by those skilled in the art that the features and advantages described above for the antibody or antigen-binding fragment thereof are equally applicable to the recombinant cell and will not be described further herein.

### Composition, Medicament, Kit

In a sixth aspect of the present disclosure, provided is a composition. According to an embodiment of the present disclosure, the composition includes the antibody or antigen-binding fragment thereof as described in the first aspect, the nucleic acid molecule as described in the second aspect, the expression vector as described in the third aspect, or the recombinant cell as described in the fifth aspect.

The composition of the present disclosure may be administered in combination with each other, or in combination with one or more other therapeutic compounds, for example, in combination with a chemotherapeutic agent. Thus, the composition may also contain a chemotherapeutic agent. The antibody or antigen-binding fragment, nucleic acid molecule, expression vector, or recombinant cell thereof of the present disclosure can also be combined with a second therapeutic agent, and the exemplary reagents of the second therapeutic agent include but are not limited to other reagents that inhibit CD112 activity (including other antibodies or antigen-binding fragments thereof, peptide inhibitors, small molecule antagonists, etc.) and/or reagents that interfere with upstream or downstream signal transduction of CD112.

In certain embodiments, the composition includes combinations that are separated in time and/or space so long as they can work together to achieve the objects of the present disclosure. For example, the components contained in the composition can be administered to the subjects as a whole or separately. When the components contained in the composition are administered separately to the subject, the individual components may be administered to the subject simultaneously or sequentially.

Usually, the antibody or antigen-binding fragment thereof is administered in an effective amount, which is sufficient to achieve the desired therapeutic and/or preventive effect, for example, an amount that results in the prevention or amelioration of symptoms associated with the disease being treated, e.g. a disease associated with CD112. The effective amount of the composition administered to the subject will depend on the type and severity of the disease, as well as on the characteristics of the individual, such as general health, age, sex, weight and tolerance to medicaments; it will also depend on the severity and type of disease, and those skilled in the art will be able to determine appropriate dosages depending on such factors, etc.

According to an embodiment of the present disclosure, the composition of the present disclosure, when introduced into an organism, can achieve the same therapeutic effect as the antibody or antigen-binding fragment thereof, nucleic acid molecule, expression vector, and recombinant cell.

It will be appreciated by those skilled in the art that the features and advantages described above for the antibody or antigen-binding fragment thereof are equally applicable to the composition and will not be described further herein.

In an eighth aspect of the present disclosure, provided is a medicament. According to an embodiment of the present disclosure, the medicament includes the antibody or antigen-binding fragment thereof as described in the first aspect, the nucleic acid molecule as described in the second aspect, the expression vector as described in the third aspect, the recombinant cell as described in the fifth aspect, or the composition as described in the sixth aspect, wherein the medicament is used for preventing and/or treating a CD112-mediated disease.

According to an embodiment of the present disclosure, the CD112-mediated disease is transplant rejection, autoimmune disease, infectious disease, or cancer.

According to an embodiment of the present disclosure, the cancer is selected from at least one of lung cancer, liver cancer, ovarian cancer, cervical cancer, skin cancer, bladder cancer, colon cancer, breast cancer, glioma, renal cancer, gastric cancer, esophageal cancer, oral squamous cell carcinoma, or head-and-neck cancer.

According to a specific embodiment of the present disclosure, the medicament may also include a pharmaceutically acceptable carrier or excipient.

It will be appreciated by those skilled in the art that the features and advantages described above for the antibody or antigen-binding fragment thereof are equally applicable to the medicament and will not be described further herein.

In a tenth aspect of the present disclosure, provided is a kit. According to an embodiment of the present disclosure, the kit includes the antibody or antigen-binding fragment thereof as described in the first aspect, the nucleic acid molecule as described in the second aspect, the expression vector as described in the third aspect, or the recombinant cell as described in the fifth aspect.

As previously described, the antibody or antigen-binding fragment of some embodiments of the present disclosure can effectively bind to human and monkey CD112 proteins, and therefore, a kit including the antibody or antigen-binding fragment can effectively detect the CD112 protein qualitatively or quantitatively. The kit provided by the present disclosure, for example, can be used for immunoblotting, immunoprecipitation, and other kits for detection involving the use of CD112 and antibody specific binding. These kits may include any one or more of the following: antagonist, anti-CD112 antibody, or medicament reference material; protein purification column; immunoglobulin affinity purification buffer; diluent for cell assay, instructions or literature, etc. The anti-CD112 antibody can be used for different types of diagnostic tests, e.g. in vitro or in vivo to detect the presence of a wide variety of diseases or medicaments, toxins, or other proteins, etc. For example, it can be used to test the serum or blood of the subject for related diseases, it can be also used to conduct scientific research, or detect CD112 protein in the sample to be tested using the kit. Such related diseases may include CD112-related diseases, such as cancer. Of course, the antibody or antigen-binding fragment provided herein can also be used for radioimmunoassay and radioimmunotherapy of the aforementioned diseases and the like. For the above application scenarios, the binding molecules are equally applicable and will not be repeated here.

According to some specific embodiments of the present disclosure, the kit may also include conventional components for detecting CD112, such as coating solution, etc.

It will be appreciated by those skilled in the art that the features and advantages described above for the antibody or antigen-binding fragment thereof are equally applicable to the kit and will not be described further herein.

### Use

In a seventh aspect of the present disclosure, provided is a use of the antibody or antigen-binding fragments thereof as described in the first aspect, the nucleic acid molecule as described in the second aspect, the expression vector as described in the third aspect, the recombinant cell as described in the fifth aspect, or the composition as described in the sixth aspect in the preparation of a medicament. According to an embodiment of the present disclosure, the medicament is used for preventing and/or treating a CD112-mediated disease. As mentioned earlier, the antibody or antigen-binding fragment of some specific embodiments of the present disclosure can effectively bind to CD112 protein. Therefore, the medicament containing an effective amount of the antibody or antigen-binding fragment or a series of substances thereof can also effectively bind to CD112 protein, having good preventive and/or therapeutic effects on CD112-mediated diseases.

According to an embodiment of the present disclosure, the CD112-mediated disease is transplant rejection, autoimmune disease, infectious disease, or cancer.

According to an embodiment of the present disclosure, the cancer is selected from at least one of lung cancer, liver cancer, ovarian cancer, cervical cancer, skin cancer, bladder cancer, colon cancer, breast cancer, glioma, renal cancer, gastric cancer, esophageal cancer, oral squamous cell carcinoma, or head-and-neck cancer.

The effective amount of the antibody or antigen-binding fragment described in the present disclosure may vary depending on the mode of administration and the severity of the disease to be treated. The selection of the optimal effective amount can be determined by those ordinary skilled in the art according to various factors (such as through clinical trials). Such factors include, but are not limited to: pharmacokinetic parameters of the active ingredient such as bioavailability, metabolism, half-life, and the like; the severity of the disease of the patient to be treated, the weight of the patient, the immune status of the patient, the route of administration, etc. For example, several separate doses may be administered daily as indicated by the exigencies of the therapeutic situation or the doses may be proportionally reduced.

In a ninth aspect of the present disclosure, provided is a use of the antibody or antigen-binding fragment thereof as described in the first aspect, the nucleic acid molecule as described in the second aspect, the expression vectors as described in the third aspect, or the recombinant cell as described in the fifth aspect in the preparation of a kit. According to an embodiment of the present disclosure, the kit is used for detecting CD112. As previously described, the antibody or antigen-binding fragments of some specific embodiments of the present disclosure can effectively bind to CD112 protein. Therefore, the antibody or antigen-binding fragments can be used to prepare a kit for detecting CD112 protein, which can effectively detect CD112 protein qualitatively or quantitatively.

According to an embodiment of the present disclosure, the kit can detect CD112 more efficiently and accurately, saving time and exploration costs for clinical treatment.

In a fifteenth aspect of the present disclosure, provided is a use of the antibody or antigen-binding fragment as described in the first aspect, the nucleic acid molecule as described in the second aspect, the expression vector as described in the third aspect, the recombinant cell as described in the fifth aspect, the combination as described in the sixth aspect, or the medicament as described in the eighth aspect in treating or preventing of a CD112-mediated related disease. As previously described, the antibody or antigen-binding fragment can effectively bind to human and monkey CD112 and can effectively treat or prevent a CD112-mediated related disease.

According to an embodiment of the present disclosure, the CD112-mediated disease is transplant rejection, autoimmune disease, infectious disease, or cancer.

According to an embodiment of the present disclosure, the cancer is selected from at least one of lung cancer, liver cancer, ovarian cancer, cervical cancer, skin cancer, bladder cancer, colon cancer, breast cancer, glioma, renal cancer, gastric cancer, esophageal cancer, oral squamous cell carcinoma, or head-and-neck cancer.

In a sixteenth aspect of the present disclosure, provided is a use of the antibody or antigen-binding fragment as described in the first aspect, the nucleic acid molecule as described in the second aspect, the expression vector as described in the third aspect or the recombinant cell as described in the fifth aspect in diagnosing or staging a CD112-mediated related disease, or evaluating prognosis of a CD112-mediated related disease. As previously described, the antibody or antigen-binding fragment, or the antibody or antigen-binding fragment expressed by the nucleic acid molecule, expression vector or recombinant cell provided in the present disclosure can effectively bind to human and monkey CD112. Therefore, the method described in the present disclosure can effectively detect the content of CD112 in a sample to be tested from a subject, and can effectively diagnose, stage and evaluate the prognosis of the related diseases mediated by CD112.

According to an embodiment of the present disclosure, the CD112-mediated disease is transplant rejection, autoimmune disease, infectious disease, or cancer.

According to a specific embodiment of the present disclosure, the cancer is at least one of lung cancer, liver cancer, ovarian cancer, cervical cancer, skin cancer, bladder cancer, colon cancer, breast cancer, glioma, renal cancer, gastric cancer, esophageal cancer, oral squamous cell carcinoma, and head-and-neck cancer.

It will be appreciated by those skilled in the art that the features and advantages described above for the antibodies or antigen-binding fragments thereof are equally applicable to the use described above and will not be described further herein.

### Method

In a fourth aspect of the present disclosure, provided is a method for preparing the antibody or antigen-binding fragment thereof as described in the first aspect. According to an embodiment of the present disclosure, the method includes: introducing the expression vector of the third aspect into cells; culturing the cells under conditions suitable for protein expression and secretion to obtain the antibody or antigen-binding fragment thereof.

The present inventors have found that preparing the antibody or the antigen-binding fragment thereof according to an embodiment of the present disclosure can cultivate the antibody or antigen-binding fragment thereof with high efficiency and high purity, and the process is simple and the cost is low.

According to some specific embodiments of the present disclosure, the cells are not particularly limited and both prokaryotic and eukaryotic cells may be used.

According to some specific embodiments of the present disclosure, the cells are eukaryotic cells.

According to some specific embodiments of the present disclosure, the eukaryotic cells are mammalian cells. According to some specific embodiments of the present disclosure, the recombinant antibody is expressed more efficiently when the cells are eukaryotic cells, such as mammalian cells.

In an eleventh aspect of the present disclosure, provided is a method for preventing and/or treating a CD112-mediated related disease. According to an embodiment of the present disclosure, the method includes administering to a subject at least one of the following: 1) the antibody or antigen-binding fragment thereof as described in the first aspect; 2) the nucleic acid molecule as described in the second aspect; 3) the expression vector as described in the third aspect; 4) the recombinant cell as described in the fifth aspect; 5) the composition described in the sixth aspect; and 6) the medicament described in the eighth aspect. As previously described, the antibody or antigen-binding fragment thereof can bind to the human and monkey CD112 protein and can effectively treat or prevent a CD112-mediated related disease, thus, the method according to an embodiment of the present disclosure can effectively treat or prevent a CD112-mediated related disease.

In a twelfth aspect of the present disclosure, provided is a method for diagnosing a CD112-mediated related disease. According to an embodiment of the present disclosure, the method includes using at least one of the following to detect the CD112 in a sample to be tested: 1) the antibody or antigen-binding fragment thereof as described in the first aspect; 2) the nucleic acid molecule as described in the second aspect; 3) the expression vector as described in the third aspect; and 4) the recombinant cell as described in the fifth aspect; determining the content of CD112 in the sample to be tested based on the detection result of CD112. The antibody or antigen-binding fragment, or the antibody or antigen-binding fragment expressed by the nucleic acid molecule, the expression vector, and the recombinant cell, as provided in the present disclosure, can effectively bind to human and monkey CD112 proteins, or the antibody or antigen-binding fragment expressed by the nucleic acid molecule, the expression vector, and the recombinant cell can effectively bind to CD112. Therefore, the method described in the present disclosure can effectively detect the content of CD112 in a sample to be tested from a subject, and can effectively diagnose a related disease caused by CD112.

According to an embodiment of the present disclosure, a content of CD112 in the sample to be tested that is not less than the minimum standard for a disease is an indication that the sample to be tested is derived from a patient suffering from a related disease caused by CD112. The value of the minimum standard can be determined by performing comparing, analyzing and verifying of the difference in the amount of CD112 in the sample to be tested from a large number of individuals suffering from a related disease caused by CD112 and a large number of healthy individuals.

According to an embodiment of the present disclosure, the sample to be tested includes at least one of the following: blood, saliva, sweat, tissue, cells, blood, serum, plasma, feces, and urine.

According to an embodiment of the present disclosure, the CD112-mediated disease is transplant rejection, autoimmune disease, infectious disease, or cancer.

According to an embodiment of the present disclosure, the cancer is at least one of lung cancer, liver cancer, ovarian cancer, cervical cancer, skin cancer, bladder cancer, colon cancer, breast cancer, glioma, renal cancer, gastric cancer, esophageal cancer, oral squamous cell carcinoma, and head-and-neck cancer.

In the thirteenth aspect of the present disclosure, provided is a method for staging a CD112-mediated related disease. According to an embodiment of the present disclosure, the method includes using at least one of the following to detect the CD112 in a sample to be tested: 1) the antibody or antigen-binding fragment thereof as described in the first aspect; 2) the nucleic acid molecule as described in the second aspect; 3) the expression vector as described in the third aspect; and 4) the recombinant cell as described in the fifth aspect; determining the content of CD112 in the sample to be tested based on the detection result of CD112. The antibody or antigen-binding fragment, or the antibody or antigen-binding fragment expressed by the nucleic acid molecule, the expression vector, and the recombinant cell, as provided in the present disclosure, can effectively bind to human and monkey CD112. Therefore, the method described in the present disclosure can effectively detect the content of CD112 in a sample to be tested from a subject, and can evaluate the stage of a related disease caused by CD112 based on the content of CD112.

According to an embodiment of the present disclosure, the content of CD112 in the sample to be tested which is not less than the standard level for disease of Stage IV tumor is an indication that the test sample is derived from a patient with disease of Stage IV tumor; the content of CD112 in the sample to be tested which lies between the standard levels for disease of Stage IV tumor and disease of Stage III tumor is an indication that the sample to be tested is derived from a patient with disease of stage III tumor; the content of CD112 in the sample to be tested being between the standard levels for disease of Stage III tumor and Stage II tumor is an indication that the sample to be tested is derived from a patient having Stage II tumor; the content of CD112 in the sample to be tested being between the standard levels for disease of Stage I and Stage II tumor is an indication that the sample to be tested is derived from a patient having Stage I tumor. It can be understood by those skilled in the art that the levels of CD112 in Stage I, II, III, and IV tumors varies depending on the type of tumor. The stage of tumors can be determined as long as the content of CD112 in the sample to be tested is compared with the corresponding standard level of CD112 at this tumor stage, or the content of CD112 in the sample to be tested is compared with the content of CD112 in the sample derived from an individual or population with known disease stage. The values of the standard levels for stage I, II, III, and IV tumors can be determined by comparing, analyzing and verifying the differences in CD112 contents in the sample to be tested between a large number of individuals with CD112-related diseases and a large number of healthy individuals.

According to an embodiment of the present disclosure, the sample to be tested includes at least one of the following: blood, saliva, sweat, tissue, cells, blood, serum, plasma, feces, and urine.

In a fourteenth aspect of the present disclosure, provided is a method for evaluating the prognosis of a CD112-mediated related disease. According to an embodiment of the present disclosure, the method includes using at least one of the following to detect the CD112 in a sample to be tested: 1) the antibody or antigen-binding fragment thereof as described in the first aspect; 2) the nucleic acid molecule as described in the second aspect; 3) the expression vector as described in the third aspect; and 4) the recombinant cell as described in the fifth aspect; determining the content of CD112 in the sample to be tested based on the detection result of CD112. As previously described, the content of CD112 has an important effect on cancer. After the treatment of an individual with the related disease, the prognosis of such a disease can be effectively evaluated by monitoring the content of CD112 in a tissue or excretion, such as peripheral blood, urine, etc., for example, by comparing the content of CD112 in a subject before and after treatment, or comparing the content of CD112 in a subject after treatment with the level of CD112 in a normal individual or an individual with a disease, etc. The antibody or antigen-binding fragment, or the antibody or antigen-binding fragment expressed by the nucleic acid molecule, the expression vector, the recombinant cell, provided in the present disclosure, can effectively bind to human and monkey CD112. Therefore, the method described in the present disclosure can effectively detect the content of CD112 in the sample to be tested from the subject, and evaluate the prognosis of related diseases caused by CD112 based on the content of CD112.

According to an embodiment of the present disclosure, the sample to be tested is derived from a patient with a CD112-mediated related disease before or after treatment.

According to an embodiment of the present disclosure, the sample to be tested includes at least one of the following: blood, saliva, sweat, tissue, cells, blood, serum, plasma, feces, and urine.

According to an embodiment of the present disclosure, the prognostic effect of a CD112-mediated related disease is determined based on the content of CD112 in a sample to be tested of a patient having a CD112-mediated related disease before or after treatment.

The embodiments of the present disclosure are described below in detail. The embodiments described below are illustrative, merely used for explaining the present disclosure, but they cannot be understood as a limitation to the present disclosure. Procedures in the embodiments without indicated specific technologies or conditions are conducted according to technologies or conditions described in the document in the art or according to the product manual. Used reagents or instruments without indicating manufacturers are all conventional products purchasable in the market.

### Example 1: Preparation of Antibody

Murine monoclonal antibody 9G11 against human CD112 was generated. Balb/c mice (9-week-old, purchased from Shanghai LAISIKE, weighing about 20 g) were immunized with CD112 extracellular domain His tag fusion protein (CD112-His) (purchased from Acro) as an antigen.

The murine monoclonal antibody 9G11 antibody against human CD112 was generated as follows: immunization mice were immunized three times with the CD112 extracellular segment His tag fusion protein (CD112-His, purchased from Acro) and Complete Freund's adjuvant and incomplete Freund's adjuvant (purchased from Sigma). Immune responses were detected after bleeding through tail vein. Serums were screened by ELISA and flow cytometry to obtain mice with anti-human CD112 immunoglobulin. The splenocytes of the mice with the highest anti-CD112 immunoglobulin were taken and then fused with mouse myeloma SP2/0 cells (ATCC NO. CRL-1581). The fused hybridoma cells were screened for antibodies to obtain murine mAb 9G11 antibody.

The candidate hybridoma cells were cultured to a total number of 10⁶. The cells were collected by centrifugation at 800 rpm for 10 min, and the total RNA was extracted with Trizol kit (Invitrogen). The total RNA was used as a template to synthesize cDNA library (Invitrogen) by reverse transcription, and the cDNA was used as a template to amplify the corresponding nucleic acid sequence of the variable region in hybridoma cells by PCR. The primer sequences used in the PCR amplification reaction were complementary to the first frame region of the antibody variable region or signal peptide region and the constant region (Larrick, J. W., et al., (1990) Scand. J. Immunol., 32, 121-128 and Coloma, J. J. et al., (1991) BioTechniques, 11, 152-156). In a 50 μL reaction system, 2 μL of cDNA, 5 μL of 10×PCR buffer, 2 μL of upstream and downstream primers (5 μmol), 2 μL of dNTP, 1 μL of Taq enzyme (Takara, Ex Taq), and 38 μL of H₂O were added, respectively. A pre-denaturation was performed for 5 min at 95° C., and the procedure was entered into the temperature cycle for PCR amplification. The reaction conditions included: denaturing at 94° C. for 30 s, annealing at 58° C. for 45 s, extension at 72° C. for 50 S for 32 cycles, then extension at 72° C. for 7 min. After the amplified products were sequenced, the heavy chain region sequence (as set forth in SEQ ID NO: 10) and light chain variable region sequence (as set forth in SEQ ID NO: 11) of mouse monoclonal antibody 9G11 were obtained.

### Example 2: Antibody Production

The specific experimental operations for antibody production were as follows: (1) ExpiCHO cells (purchased from Thermo Fisher) were cultured using ExpiCHO Expression Medium (purchased from Thermo Fisher), and a cell concentration was adjusted to 6×10⁶/mL to obtain an ExpiCHO cell solution. (2) pcDNA3.4 vector (entrusted to Nanjing Ginscript to synthesize) containing an antibody heavy chain and an antibody light chain was added to 2 mL of OptiSFM medium (purchased from Thermo Fisher) according to a ratio of 1:1 to obtain solution A. (3) 160 μL of ExpiFectamineCHO transfection reagent (purchased from Thermo Fisher) was added to 2 mL of OptiSFM medium (purchased from Thermo Fisher) to obtain Solution B. (4) the solution A and solution B were then mixed to obtain a transfection mixture and the whole transfection mixture was added to the 50 mL of ExpiCHO cell solution within 5 minutes. (5) 8 mL of Feed, and 300 μL of Enhancer (purchased from Thermo Fisher) were added after 1 day of culture at 37° C., 5% CO₂, and the culture supernatant was harvested after 9 days of culture at 32° C. and 5% CO₂, and 8 mL of Feed was added on Day 5. (6) the antibody of interest was obtained by affinity purification from the culture supernatant using a Protein A purification column (purchased from Nano-micro).

### Example 3: ELISA Binding Experiment of Murine CD112 Antibody

The ELISA experiment was used to determine the binding property of the CD112 antibody (9G11 antibody). The CD112 extracellular region His tag fusion protein was coated into a 96-well plate. The signal intensity after antibody addition was used to determine the binding characteristic of antibody and CD112 protein.

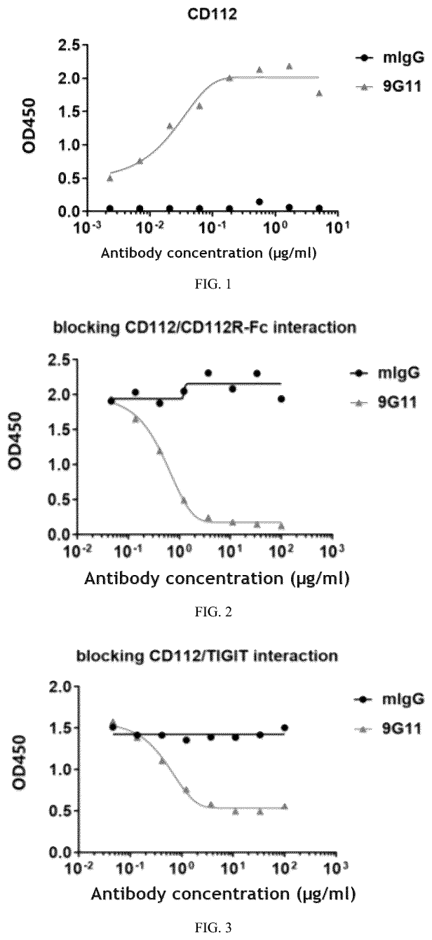

The CD112-His protein was diluted to 2 μg/ml with PBS buffer and added to a 96-well plate in a volume of 100 μl/well and left overnight at 4° C. The PBS buffer solution was sucked off from the 96-well plate. The plate was washed 6 times with PBST (pH 7.2, PBS containing 0.1 vol % of Tween 20) buffer solution. 200 μL of PBS containing 10% BSA was added per well and incubated at 37° C. for 2 hours for blocking. The blocking solution was removed and the plate was washed 6 times with PBST. Then, 100 μl of the CD112 murine antibody 9G11 to be tested (heavy chain sequence is SEQ ID NO: 16, and light chain sequence is SEQ ID NO: 17) diluted gradient-wise in a PBST containing 0.05% BSA (maximum concentration 5 μg/ml, 8 gradients, diluted 3-fold for each gradient) was added per well and incubated for 1 h at 37° C. The reaction system was removed. The plate was washed 6 times with PBST. 100 μL of HRP (horseradish peroxidase)-labeled anti-mouse IgG antibody secondary (purchased from Southern biotech) antibody diluted with PBST containing 0.05% BSA (diluted at 1:5000) per well and incubated for 1 h at 37° C. The plate was washed 6 times with PBST, and 80 μL of TMB (tetramethylbenzidine) was added per well. After 3 min of incubation at room temperature, 80 μL of 4M sulfuric acid was added per well to terminate the reaction. The absorbance was read at 450 mm with a microplate reader. The results, as set forth in FIG. 1, indicate that the CD112 antibody 9G11 antibody of the present disclosure can bind to the CD112 protein.

(SEQ ID NO: 16)
DVKLVESGGGLVKLGGSLKLSCAASGFTFSSYYMSWVRQTPEKRL
ELVAAINSQGGSTYYPDTVKGRFTISRDNAKNTLYLQMSSLKSE
DTGLYYCARSDYDWAWFAYWGQGTLVTVSAAKTTAPSVYPLAPVC
GDTTGSSVTLGCLVKGYFPEPVTLTWNSGSLSSGVHTFPAVLQSD
LYTLSSSVTVTSSTWPSQSITCNVAHPASSTKVDKKIEPRGPTIK
PCPPCKCPAPNLLGGPSVFIFPPKIKDVLMISLSPIVTCVVVDVS
EDDPDVQISWFVNNVEVHTAQTQTHREDYNSTLRVVSALPIQHQD
WMSGKEFKCKVNNKDLPAPIERTISKPKGSVRAPQVYVLPPPEEE
MTKKQVTLTCMVTDFMPEDIYVEWTNNGKTELNYKNTEPVLDSDG
SYFMYSKLRVEKKNWVERNSYSCSVVHEGLHNHHTTKSFSRTPGK.
(SEQ ID NO: 17)
DIVMSQSPSSLAVSVGEKVAMSCKSSQSLLYSSNQKNYLAWYQQK
PGQSPKLLIYWASTRESGVPDRFTGSGSGTDFTLTISSVKAEDLA
VYYCQQYYRYPPTFGAGTKLELKRADAAPTVSIFPPSSEQLTSGG
ASVVCFLNNFYPKDINVKWKIDGSERQNGVLNSWTDQDSKDSTYS
MSSTLTLTKDEYERHNSYTCEATHKTSTSPIVKSFNRNEC.

### Example 4: Assay of Murine CD112 Antibody for Blocking Ability

The ELISA experiment was used to determine the blocking characteristic of the murine antibody CD112 9G11 antibody. Human CD112 extracellular region His tag fusion protein was coated into a 96-well plate, and the signal intensity after the addition of 9G11 antibody and CD112R-Fc-Biotin, TIGIT-Biotin, and CD226-Biotin (purchased from Acro) was used to determine the effect of 9G11 antibody on the binding of CD112 to CD112R, TIGIT, and CD226.

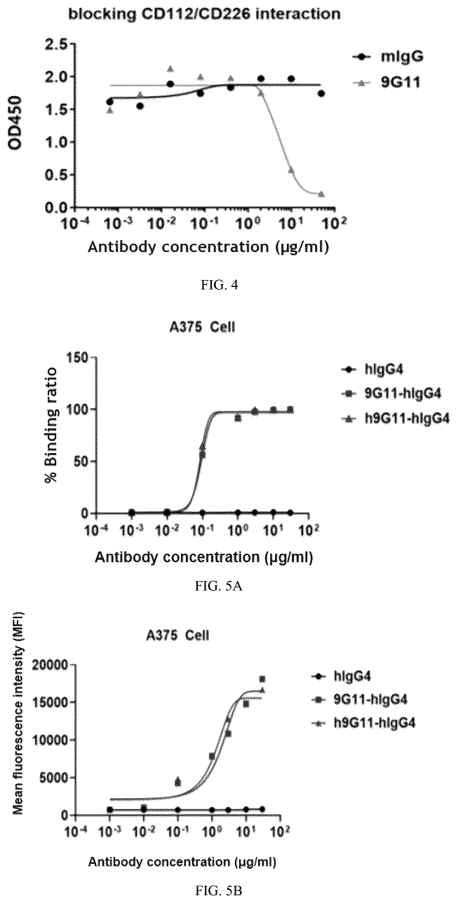

The human CD112-His protein (purchased from Acro) was diluted to 2 μg/ml with PBS buffer and added to a 96-well plate in a volume of 100 μl/well and left overnight at 4° C. The PBS buffer solution was sucked off from the 96-well plate. The plate was washed 6 times with PBST (pH 7.2, PBS containing 0.1 vol % of Tween 20) buffer solution. 200 μL of PBS containing 10% BSA was added per well and incubated at 37° C. for 2 hours for blocking. The blocking solution was removed and the plate was washed 6 times with PBST. 50 μL of CD112R-Fc-Biotin (2 μg/ml), TIGIT-Biotin (25 μg/ml), or CD226-Biotin (50 μg/ml) (purchased from Acro) diluted to an appropriate concentration with PBST containing 0.05% BSA was added per well. The wells containing CD112R-Fc-Biotin, TIGIT-Biotin, or CD226-Biotin were added with 50 μL of CD112 antibody 9G11 to be tested (heavy chain sequence is SEQ ID NO: 16, and light chain sequence is SEQ ID NO: 17) diluted gradient-wise in a PBST containing 0.05% BSA or control mIgG (purchased from Sino Biological Inc.), respectively, in a total volume of 100 μL, followed by incubation for 1 h at 37° C. The reaction system was removed. The plate was washed 6 times with PBST. 100 μL of HRP (horseradish peroxidase)-labeled Streptavidin secondary (purchased from Southern Biotech) antibody diluted with PBST containing 0.05% BSA (diluted at 1:5000) per well and incubated for 1 h at 37° C. The plate was washed 6 times with PBST, and 80 μL of TMB (tetramethylbenzidine) was added per well. After 3 min of incubation at room temperature, 80 μL of 4M sulfuric acid was added per well to terminate the reaction. The absorbance was read at 450 mm with a microplate reader. The results are shown in FIGS. 2, 3, and 4, demonstrating that the CD112 antibody 9G11 antibody of the present disclosure can block CD112 from binding to its receptors CD112R, TIGIT, and CD226.

### Example 5: Experiment on Humanization of Mouse Antibody

With reference to the light chain variable sequence and the heavy chain variable sequence of the CD112 antibody 9G11 antibody, the humanized template that best matches the non-CDR region thereof was selected. The CDR region of the murine antibody was transplanted onto the selected humanized template to replace the CDR region of the humanized template to obtain a humanized antibody. Then, based on the three-dimensional structure of the murine antibody, reverse mutations were performed on the embedded residues, the residues having a direct interaction with the CDR region, and the residues having an important influence on the conformation of VL and VH to obtain a humanized antibody h9G11 antibody. The sequence of the heavy chain variable region of humanized CD112 antibody h9G11 antibody is as set forth in SEQ ID NO: 12, and the sequence of the light chain variable region is as set forth in SEQ ID NO: 13.

### Example 6: Flow Cytometry Binding Experiment of Chimeric CD112 Antibody 9G11-hIgG4 with Humanized CD112 Antibody h9G11-hIgG4

Flow cytometry experiment was used to detect the binding characteristics of chimeric CD112 antibody 9G11-hIgG4 (prepared by the method described in Example 2) and humanized CD112 antibody h9G11-hIgG4. Chimeric CD112 antibody 9G11-hIgG4 and humanized CD112 antibody h9G11-hIgG4 were added to A-375 melanoma cells and A-549 non-small cell lung cancer cells. The signal intensity after the addition of antibody was used to determine the binding characteristics of the antibody and CD112.

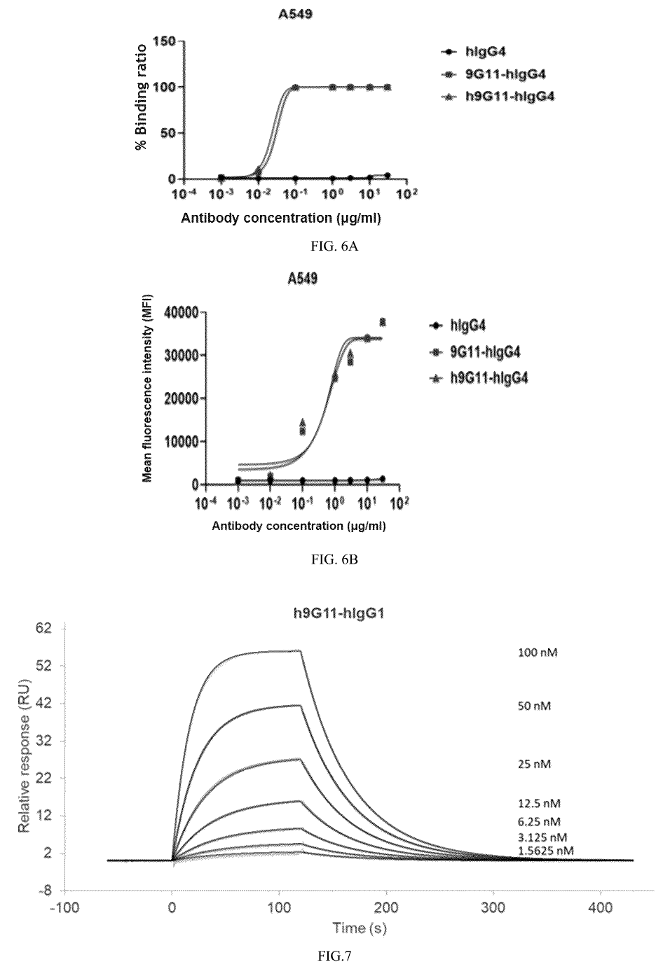

The cancer cells were diluted with PBS to 2×10⁶/mL and were added to a 1.5 mL EP tube at 100 μL/tube. Goat serum at 10 μL/tube was added thereto and was blocked at 4° C. for 30 min. The human murine chimeric CD112 antibody 9G11-hIgG4 (heavy chain sequence is SEQ ID NO: 18, and light chain sequence is SEQ ID NO: 19), the humanized CD112 antibody h9G11-hIgG4 (heavy chain sequence is SEQ ID NO: 20, and light chain sequence is SEQ ID NO: 21), and the control hIgG4 (purchased from Biointron) diluted gradient-wise (30, 10, 3, 1, 0.1, 0.01, 0.001 μg/mL) were added and incubated at 4° C. for 30 min. The EP tube was filled with 1 mL of PBS and centrifuged at 3500 rpm at 4° C. for 5 min. The supernatant was discarded and the pellet was washed once more with PBS. After centrifugation, the supernatant was discarded. The cells were resuspended with 100 μL/tube of PBS, to which 1 μL/tube of Alexa-647-labeled anti-human IgG-antibody secondary antibody (purchased from Jackson lab) was added, and incubated at 4° C., protected from light, for 30 min. The cell-containing pellet was washed twice with PBS and the supernatant was discarded after centrifugation. The cells were resuspended with 200 μL/tube PBS and detected by the flow cytometry. The results are as shown in FIGS. 5A, 5B, 6A and 6B, showing that the chimeric CD112 antibody 9G11-hIgG4 and humanized CD112 antibody h9G11-hIgG4 of the present disclosure can bind to A-375 and A-549 tumor cells, with comparative binding ability.

(SEQ ID NO: 18)
DVKLVESGGGLVKLGGSLKLSCAASGFTFSSYYMSWVRQTPEKRL
ELVAAINSQGGSTYYPDTVKGRFTISRDNAKNTLYLQMSSLKSED
TGLYYCARSDYDWAWFAYWGQGTLVTVSAASTKGPSVFPLAPCSR
STSESTAALGCLVKDYFPEPVTVSWNSGALTSGVHTFPAVLQSSG
LYSLSSVVTVPSSSLGTKTYTCNVDHKPSNTKVDKRVESKYGPPC
PPCPAPEFLGGPSVFLFPPKPKDTLMISRTPEVTCVVVDVSQEDP
EVQFNWYVDGVEVHNAKTKPREEQFNSTYRVVSVLTVLHQDWLNG
KEYKCKVSNKGLPSSIEKTISKAKGQPREPQVYTLPPSQEEMTKN
QVSLTCLVKGFYPSDIAVEWESNGQPENNYKTTPPVLDSDGSFFL
YSRLTVDKSRWQEGNVFSCSVMHEALHNHYTQKSLSLSLGK.
(SEQ ID NO: 19)
DIVMSQSPSSLAVSVGEKVAMSCKSSQSLLYSSNQKNYLAWYQQK
PGQSPKLLIYWASTRESGVPDRFTGSGSGTDFTLTISSVKAEDLA
VYYCQQYYRYPPTFGAGTKLELKRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGEC.
(SEQ ID NO: 20)
EVQLVESGGGLVQPGGSLRLSCAASGFTFSSYYMSWVRQAPGKRL
ELVAAINSQGGSTYYPDTVKGRFTISRDNAKNTLYLQMNSLRAE
DTAVYYCARSDYDWAWFAYWGQGTLVTVSSASTKGPSVFPLAPCS
RSTSESTAALGCLVKDYFPEPVTVSWNSGALTSGVHTFPAVLQSS
GLYSLSSVVTVPSSSLGTKTYTCNVDHKPSNTK VDKRVESKYGP
PCPPCPAPEFLGGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLTCLVKGFYPSDIAVEWESNGQPENNYKTTPPVLDSDGSF
FLYSRLTVDKSRWQEGNVFSCSVMHEALHNHYTQKSLSLSLGK.
(SEQ ID NO: 21)
DIVMTQSPSSLAVSLGERATINCKSSQSLLYSSNQKNYLAWYQQK
PGQPPKLLIYWASTRESGVPDRFSGSGSGTDFTLTISSLQAEDVA
VYYCQQYYRYPPTFGGGTKLEIKRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGEC.

### Example 7: Antibody Affinity Maturation

During an affinity maturation process of the natural antibody, somatic high-frequency mutations are mainly concentrated in the CDR region. Single point saturation mutation was performed at each site in the CDR region by in vitro experiments to obtain sufficient mutation diversity without disruption of the protein structure. Such a route can realize in-vitro reproduction of the somatic hypermutation of the natural antibody in vivo with the highest similarity.

Each amino acid site of the CDR region was subjected to the single point saturation mutation to construct a deviation-free single point saturation mutation plasmid library of the maternal antibody. Mutation sites with enhanced specific binding to an antigen were screened out with ELISA. Then, these sites were subjected to combinatorial screening to obtain candidate antibody mutation sequences.

Through this technical route, the parent humanized antibody h9G11 (heavy chain variable region SEQ ID NO: 12 and light chain variable region SEQ ID NO: 13) is affinity mature to obtain the high affinity CD112 monoclonal antibody h9G11ZH21, and the sequences of its heavy chain variable region (SEQ ID NO: 14) and light chain variable region (SEQ ID NO: 15). A comparison of the CDR sequences of the parental humanized antibody h9G11 and the affinity mature antibody h9G11ZH21 is shown in Table 1.

TABLE 1

| | HCDR1 | HCDR2 | HCDR3 | LCDR1 | LCDR2 | LCDR3 |
|---|---|---|---|---|---|---|
| h9G11 | GFTFSSYY | INSQGGST | ARSDYDWAWFAY | QSLLYSSNQKNY | WAS | QQYYRYPPT |
| h9G11ZH21 | GFTFSSYM | INSQGGST | ARSDYDWAWYAY | QSLLYSSNQKNY | WAN | QQYYRYPPT |

### Example 8: Affinity Assay of Antibody

Biacore, as a method for analyzing biomolecular interaction based on an optical surface plasmon resonance (SPR), can not only detect specific binding between an antigen and an antibody, but also obtain very important data in research and development of medicaments, such as a binding rate constant (Ka) among molecules, a dissociation rate constant (Kd), and an equilibrium dissociation constant (KD), thereby calculating the affinity of the antibody.

In a Biacore 8K (Cytiva) system, the antibody was diluted to a concentration of 10 μg/mL with running buffer (HBS-EP), and the antibody was conjugated onto a protein A (Cytiva, 29127556) chip at a flow rate of 10 μl/min. Kinetics and affinity data of the binding of the antigen and the antibody were detected at a flow rate of 30 μl/min; binding period was 120 s; and dissociation time was 800 s.

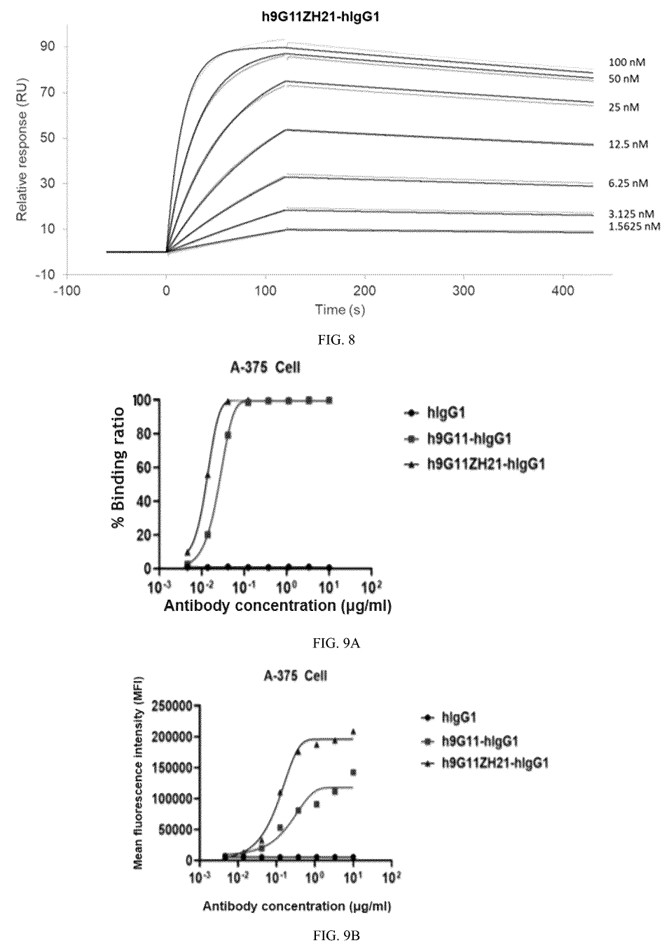

The kinetic and affinity data for the binding of the parental antibody h9G11 and the affinity mature antibody h9G11ZH21 to CD112 were examined and the results are shown in FIGS. 7 and 8, and Table 2. The affinity of the affinity mature antibody h9G11ZH21 to CD112 was substantially increased by about 75-fold compared to the parental antibody h9G11.

TABLE 2

| Ab | ka (1/Ms) | kd(1/s) | KD(M) |
|---|---|---|---|
| h9G11 | 4.39E+05 | 2.26E−02 | 5.14E−08 |
| h9G11ZH21 | 6.22E+05 | 4.22E−04 | 6.78E−10 |

Notes: ka represents a binding rate constant (the larger the value is, the higher the affinity is); kd represents a dissociation rate constant (the smaller the number is, the higher the affinity is), reflecting an affinity of a compound to a target; and KD represents kd/ka, which is the equilibrium dissociation constant (an affinity constant), wherein the smaller the KD is, the less the dissociation is, and the higher the affinity is.

### Example 9: Flow Cytometry Binding Experiment Between Humanized Antibody h9G11-hIgG1 and Affinity Mature Antibody h9G11ZH21-hIgG1

Flow cytometry experiment was used to detect the binding characteristics of humanized antibody h9G11-hIgG1 and affinity mature antibody h9G11ZH21-hIgG1. The humanized antibody h9G11-hIgG1 and affinity mature antibody h9G11ZH21-hIgG1 were added to A-375 melanoma cells, MDA-MB-231 breast cancer cells, and the signal intensity after the addition of antibody was used to determine the binding characteristics of the antibody and CD112.

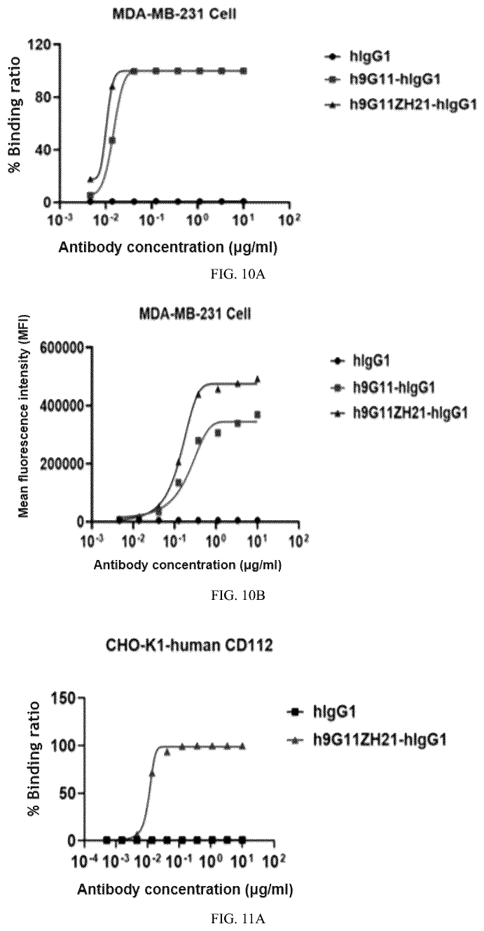

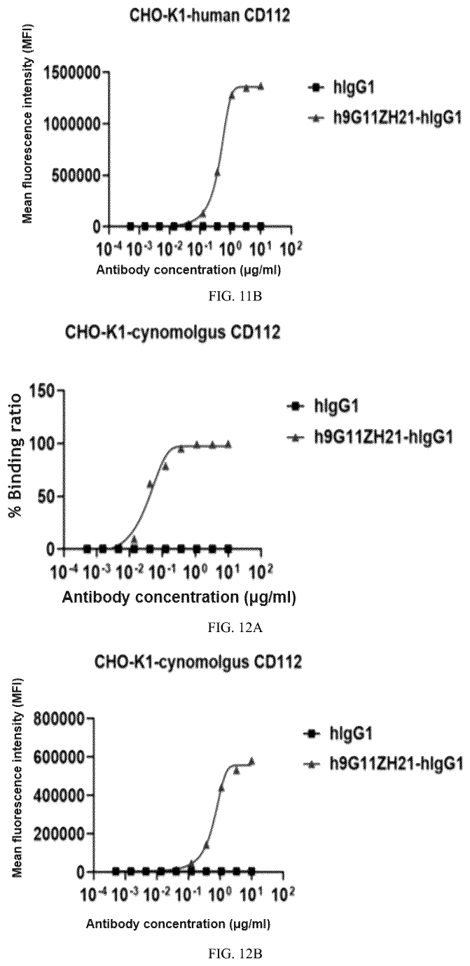

The cancer cells were diluted with PBS to 2×10⁶/mL and were added to a 1.5 mL EP tube at 100 μL/tube. Goat serum at 10 μL/tube was added thereto and was blocked at 4° C. for 30 min. The humanized antibody h9G11-hIgG1 (heavy chain sequence is SEQ ID NO: 22, and light chain sequence is SEQ ID NO: 21), affinity mature antibody h9G11ZH21-hIgG1 (heavy chain sequence is SEQ ID NO: 23, and light chain sequence is SEQ ID NO: 24), control hIgG1 (purchased from Biointron) diluted gradient-wise (up to 10 μg/mL, 8 gradients, diluted 3-fold for each gradient) were added and incubated at 4° C. for 30 min. The EP tube was added with 1 mL of PBS, and centrifuged at 3500 rpm at 4° C. for 5 min. The supernatant was discarded and the pellet was washed once more with PBS. After centrifugation, the supernatant was discarded. The cells were resuspended with 100 μL/tube of PBS, to which 1 μL/tube of Alexa-647-labeled anti-human IgG-antibody secondary antibody (purchased from Jackson lab) was added, and incubated at 4° C., protected from light, for 30 min. The cell-containing pellet was washed twice with PBS and the supernatant was discarded after centrifugation. Cells were resuspended using 200 μL/tube PBS and detected by flow cytometry. The results are shown in FIGS. 9A, 9B, 10A and 10B, showing that the affinity mature antibody h9G11ZH21-hIgG1 of the present disclosure has stronger binding ability with A-375 melanoma cells and MDA-MB-231 breast cancer cells than the humanized antibody h9G11-hIgG1.

(SEQ ID NO: 22)
EVQLVESGGGLVQPGGSLRLSCAASGFTFSSYYMSWVRQAPGKRL
ELVAAINSQGGSTYYPDTVKGRFTISRDNAKNTLYLQMNSLRAE
DTAVYYCARSDYDWAWFAYWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSREE
MTKNQVSLTCLVKGFYPSDIAVEWESNGQPENNYKTTPPVLDSDG
SFFLYSKLTVDKSRWQQGNVFSCSVMHEALHNHYTQKSLSLSPGK.
(SEQ ID NO: 23)
EVQLVESGGGLVQPGGSLRLSCAASGFTFSSYMMSWVRQAPGKRL
ELVAAINSQGGSTYYPDTVEGRFTISRDNAKNTLYLQMNSLRAE
DTAVYYCARSDYDWAWYAYWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKK VEPKSCD
KTHTCPPCPAPELLGGPSVFLFPPKPKDTLMISRTPEVTCVVVDV
SHEDPEVKFNWYVDGVEVHNAKTKPREEQYNSTYRVVSVLTVLHQ
DWLNGKEYKCKVSNKALPAPIEKTISKAKGQPREPQVYTLPPSRE
EMTKNQVSLTCLVKGFYPSDIAVEWESNGQPENNYKTTPPVLDSD
GSFFLYSKLTVDKSRWQQGNVFSCSVMHEALHNHYTQKSLSLSPG
K.
(SEQ ID NO: 24)
DIVMTQSPSSLAVSLGERATINCKSSQSLLYSSNQKNYLAWYQQK
PGQPPKLLIYWANTRESGVPDRFSGSGSGTDFTLTISSLQAEDV
AVYYCQQYYRYPPTFGGGTKLEIKRTVAAPSVFIFPPSDEQLKSG
TASVVCLLNNFYPREAKVQWKVDNALQSGNSQESVTEQDSKDSTY
SLSSTLTLSKADYEKHKVYACEVTHQGLSSPVTKSFNRGEC.

### Example 10: Flow Cytometry Binding Experiment for Affinity Mature Antibody

Flow cytometry experiment was used to detect the binding characteristic of affinity mature antibody h9G11ZH21-hIgG1. Human and monkey CD112 (CHO-K1 human CD112, CHO-K1 cynomolgus CD112) were overexpressed in CHO-K1 cells and the signal intensity after the addition of antibody was used to determine the binding characteristics of the antibody with human and monkey CD112.

Preparation of CHO-K1 human CD112 and CHO-K1 cynomolgus CD112: HEK293T cells were cultured overnight in DMEM medium without double antibody on a 6-well plate with 5×10⁵ cells per well. The medium was discarded before transfection and 1 mL of fresh DMEM medium without double antibody was added. The coding sequence (SEQ ID NO: 27) of human CD112 protein (SEQ ID NO: 25) was inserted between the enzyme cleavage sites EcoRI and BamHI of pLVX-EF1a-human CD112-IRES-puro (entrusted to Nanjing Ginscript to synthesize, the pLVX-EF1a-IRES-puro vector (purchased from Takara)), or the coding sequence (SEQ ID NO: 28) monkey CD112 protein (SEQ ID NO: 26) was inserted between the enzyme cleavage sites EcoRI and BamHI of pLVX-EF1a-IRES-puro vector of pLVX-EF1a-human CD112-IRES-puro (entrusted to Nanjing Ginscript to synthesize, pLVX-EF1a-IRES-puro vector (purchased from Takara)), pMD 2G (purchased from Takara) and psPAX2 (purchased from Takara) vectors (total of 3 μg) were added to 200 μL serum-free DMEM medium in a ratio of 2:1:1, then 12 μg of polyetherimide (PEI, available from Polysciences) was added. After mixing well, the mixture was stood for 16 min. Then the whole liquid was added to a 6-well plate plated with HEK293T cells. After 6 h of culture, the medium was discarded and a fresh complete DMEM medium was added for culture. After 48 h of transfection, the cell culture supernatant was collected and passed through a 0.45 μm filter (purchased from Millipore) to obtain the virus supernatant. All virus supernatant was added to 6-well plates containing 1×10⁴ CHO-K1 cells, added with polybrene (purchased from Sigma) at a final concentration of 4 μg/mL, and incubated for 12 h. The supernatant was then discarded and a fresh complete DMEM medium was added. The resulting cells were CHO-K1-human CD112 and CHO-K1-cynomolgus CD112 cells.

CHO-K1 human CD112 and CHO-K1 cynomolgus CD112 cells were diluted to 2×10⁶/mL with PBS and was added to a 1.5 mL EP tube at 100 μL/tube. Goat serum at 10 μL/tube was added thereto and was blocked at 4° C. for 30 min. The affinity mature antibody h9G11ZH21-hIgG1 (heavy chain sequence is SEQ ID NO: 23, and light chain sequence is SEQ ID NO: 24) and control hIgG1 (purchased from Biointron) diluted gradient-wise (up to 10 μg/ml, 10 gradients, diluted 3-fold for each gradient) were added and incubated at 4° C. for 30 min. The EP tube was filled with 1 mL of PBS and centrifuged at 3500 rpm at 4° C. for 5 min. The supernatant was discarded and the pellet was washed once more with PBS. After centrifugation, the supernatant was discarded. The cells were resuspended with 100 μL/tube of PBS, to which 1 μL/tube of Alexa-647-labeled anti-human Fc-antibody secondary antibody (purchased from Jackson lab) was added, and incubated at 4° C., protected from light, for 30 min. The cell-containing pellet was washed twice with PBS and the supernatant was discarded after centrifugation. The cells were resuspended with 200 μL/tube PBS and detected by the flow cytometry. The results are shown in FIGS. 11A, 11B, 12A and 12B, further showing that the antibody h9G11ZH21-hIgG1 of the present disclosure can bind to human and monkey CD112.

(SEQ ID NO: 25)
MARAAALLPSRSPPTPLLWPLLLLLLLETGAQDVRVQVLPEVRGQ
LGGTVELPCHLLPPVPGLYISLVTWQRPDAPANHQNVAAFHPKMG
PSFPSPKPGSERLSFVSAKQSTGQDTEAELQDATLALHGLTVEDE
GNYTCEFATFPKGSVRGMTWLRVIAKPKNQAEAQKVTFSQDPTTV
ALCISKEGRPPARISWLSSLDWEAKETQVSGTLAGTVTVTSRFTL
VPSGRADGVTVTCKVEHESFEEPALIPVTLSVRYPPEVSISGYDD
NWYLGRTDATLSCDVRSNPEPTGYDWSTTSGTFPTSAVAQGSQLV
IHAVDSLFNTTFVCTVTNAVGMGRAEQVIFVRETPNTAGAGATGG
IIGGIIAAIIATAVAATGILICR QQRKEQTLQGAEEDEDLEGPP
SYKPPTPKAKLEAQEMPSQLFTLGASEHSPLKTPYFDAGASCTEQ
EMPRYHELPTLEERSGPLHPGATSLGSPIPVPPGPPAVEDVSLDL
EDEEGEEEEEYLDKINPIYDALSYSSPSDSYQGKGFVMSRAMYV.
(SEQ ID NO: 26)
MARAVALLPSRSPPTPLLWPLLLLLLRKTGAQDVRVQVLPEVRGQ
LGGTVELPCHLLPPVPGLYISLVTWQRPDAPPDHQNVAAFHPKMG
PSFPSPKPGSQRLSFVSAKQSTRQDTEAELQDATLALRGLTVEDE
GNYTCEFATFPKGSVRGMTWLRVIAKPQNHAEAQEVTFSQDPVPV
ARCISKEGRPPARISWLSSLDWEAKETQVSGTLAGTVTVTSRFTL
VPSGRADGVTVTCK VEHESFEEPALIPVTLSVRYPPEVSISGYD
DNWYLGRTDATLSCDVHSNPEPTGYDWSTTSGIFPTSAVAQGSQL
VIHAVDSLFNTTFVCTVTNAVGMGRAEQVIFVRETPNTAGAGATG
GIIGGIIAAIIATAVAATGILICR QQRKEQTLQGAEEDEDLEGP
PSYKPPTPKAKLEEQEMPSQLFTLGASEHSPLKTPYFDAGASCTE
QEMPRYHELPTLEERSGPLHPGATSLGSPIPVPPGPPVVEDVSLD
LEDEEGEEEEEYLDKINPVYDALSYSSPSDSYQGKGFVMSRAMYV
(SEQ ID NO: 27)
ATGGCCCGCGCTGCAGCCCTTTTGCCTTCCCGCTCTCCCCCTACA
CCACTCCTGTGGCCACTGCTTCTGCTGCTCCTGCTTGAGACAGGA
GCCCAGGACGTCAGGGTGCAGGTGCTGCCTGAAGTGAGGGGCCAG
CTGGGGGGTACAGTAGAGTTGCCATGCCACCTGCTGCCACCAGTC
CCTGGCCTGTACATATCTCTGGTCACCTGGCAGCGTCCCGATGCA
CCCGCTAATCACCAGAACGTGGCAGCCTTTCACCCCAAGATGGGT
CCAAGCTTCCCTAGTCCCAAACCTGGGTCAGAGCGGCTCTCCTTC
GTTAGTGCTAAACAGAGTACAGGACAGGACACCGAAGCCGAACTG
CAGGACGCTACTCTCGCTCTTCATGGTTTGACAGTGGAGGACGAA
GGCAACTACACCTGTGAGTTCGCTACATTTCCTAAAGGTTCCGTG
CGGGGTATGACCTGGTTGAGGGTGATTGCAAAGCCCAAGAACCAG
GCCGAGGCCCAGAAAGTTACATTTTCACAGGATCCCACTACCGTC
GCACTGTGCATTAGTAAGGAGGGGAGGCCACCTGCACGAATCAGT
TGGCTGTCCAGCCTTGACTGGGAGGCCAAGGAAACCCAAGTTAGC
GGAACTCTGGCTGGGACCGTCACAGTGACAAGCAGGTTTACTCTC
GTCCCCTCAGGGAGAGCAGATGGTGTGACAGTGACATGTAAGGTT
GAGCATGAATCCTTTGAGGAGCCAGCCTTGATTCCCGTTACTTTG
AGCGTCCGGTATCCTCCCGAGGTGTCAATTTCAGGCTATGATGAC
AACTGGTATCTCGGTCGGACAGATGCCACACTGAGCTGTGATGTG
CGAAGTAACCCCGAACCTACCGGCTATGACTGGAGTACCACATCT
GGTACCTTCCCAACCTCTGCCGTGGCACAGGGCAGCCAGCTCGTC
ATACATGCCGTTGACTCACTGTTCAACACTACATTTGTGTGCACT
GTGACTAACGCCGTGGGAATGGGACGGGCCGAACAGGTTATCTTC
GTGCGCGAAACCCCAAATACAGCCGGGGCAGGGGCTACTGGCGGC
ATCATCGGGGGTATCATTGCCGCTATCATTGCCACCGCCGTCGCT
GCTACAGGTATTCTGATATGTCGACAGCAACGGAAGGAACAGACA
CTGCAGGGAGCCGAGGAGGATGAGGACCTTGAGGGCCCCCCTTCT
TACAAGCCTCCAACACCCAAGGCTAAGCTGGAAGCCCAAGAGATG
CCTAGCCAGCTGTTTACTCTCGGCGCCAGCGAGCACTCACCCCTG
AAAACCCCTTATTTCGATGCTGGCGCATCCTGCACAGAACAAGAG
ATGCCCCGGTACCACGAACTGCCTACCTTGGAGGAAAGATCCGGC
CCATTGCATCCTGGCGCAACCTCTCTCGGCAGTCCAATCCCTGTA
CCTCCTGGCCCCCCTGCAGTGGAGGATGTTTCCCTGGATCTGGAG
GACGAAGAGGGCGAGGAGGAGGAAGAGTATTTGGACAAGATCAAC
CCTATCTACGACGCCCTCAGCTATTCCTCTCCATCTGACTCCTAC
CAGGGTAAAGGCTTCGTCATGAGCCGCGCTATGTATGTG.
(SEQ ID NO: 28)
ATGGCTCGAGCTGTCGCACTTCTCCCCTCCAGATCTCCCCCAACC
CCCCTGCTGTGGCCTCTTTTGCTTCTGCTTCTTCGTAAAACAGG
CGCTCAGGACGTTCGGGTCCAGGTGCTCCCCGAAGTGAGAGGTCA
GCTCGGAGGGACAGTGGAGCTTCCCTGCCACCTCCTTCCACCTGT
TCCCGGGCTGTATATCTCCCTTGTGACCTGGCAAAGGCCTGATGC
ACCCCCTGACCACCAAAATGTGGCCGCTTTCCACCCAAAAATGGG
CCCCTCATTTCCCAGCCCAAAGCCAGGAAGCCAGAGACTGTCCTT
CGTGAGCGCTAAGCAGTCTACCAGGCAGGATACCGAAGCTGAGCT
GCAGGATGCTACTCTTGCATTGCGGGGACTGACAGTTGAGGACGA
AGGGAACTACACATGCGAGTTTGCAACATTCCCAAAAGGTTCCGT
GAGGGGCATGACTTGGCTTAGGGTTATTGCTAAACCTCAGAATCA
CGCTGAGGCCCAGGAGGTGACTTTTTCCCAAGATCCTGTGCCCGT
CGCTAGGTGTATCTCTAAGGAGGGACGGCCCCCAGCCCGGATCTC
ATGGCTTTCAAGTCTCGATTGGGAGGCTAAGGAGACTCAAGTCAG
TGGGACACTCGCCGGAACAGTTACCGTAACCAGCCGATTCACACT
CGTTCCAAGCGGACGCGCAGATGGGGTCACCGTGACCTGCAAGGT
CGAACATGAAAGTTTTGAGGAACCAGCCCTGATACCCGTGACCCT
GTCAGTTCGGTACCCACCCGAGGTGTCCATTAGCGGTTACGACGA
TAACTGGTACCTTGGCCGCACTGATGCAACCCTGTCATGCGATGT
GCACTCCAATCCAGAACCAACTGGATACGATTGGTCCACAACTAG
CGGCATCTTCCCAACCTCTGCCGTGGCTCAAGGTTCCCAGCTCGT
GATCCACGCCGTTGACAGTCTGTTCAACACCACCTTCGTGTGTAC
TGTTACTAACGCCGTCGGAATGGGACGCGCAGAACAGGTCATCTT
TGTGCGAGAAACCCCTAACACTGCTGGAGCTGGGGCCACAGGAGG
GATCATTGGTGGAATTATCGCAGCCATCATTGCTACAGCCGTAGC
CGCTACCGGCATTCTGATTTGTAGACAGCAGAGAAAGGAACAAAC
ACTGCAGGGTGCCGAGGAGGACGAGGATCTGGAGGGGCCTCCTTC
ATACAAACCACCTACTCCAAAGGCCAAATTGGAGGAGCAGGAGAT
GCCCTCACAGTTGTTTACCTTGGGGGCCTCCGAACACTCTCCTTT
GAAGACACCATACTTCGACGCCGGGGCTTCCTGTACAGAACAGGA
GATGCCACGGTACCATGAGTTGCCCACCTTGGAAGAGAGGTCAGG
ACCCCTGCATCCTGGGGCTACCTCACTGGGGTCTCCTATACCAGT
ACCTCCAGGCCCTCCAGTGGTCGAGGATGTAAGTCTGGACTTGGA
GGACGAGGAAGGGGAGGAGGAGGAAGAGTACCTGGATAAAATTAA
TCCCGTGTACGACGCACTCTCTTACTCCTCCCCATCCGACAGCTA
CCAGGGCAAGGGATTTGTGATGTCTAGGGCAATGTACGTG.

### Example 11: Detection of the Blocking Ability of Affinity Mature CD112 Antibody (ELISA Method)

The ELISA experiment was used to detect the blocking characteristic of affinity mature CD112 antibody. Human CD112 extracellular region His tag fusion protein was coated into a 96-well plate, and the signal intensity after the addition of antibody and CD112R-Fc-Biotin and CD226-Biotin (purchased from Acro) was used to determine the effect of antibody on the binding of CD112 to CD112R and CD226.

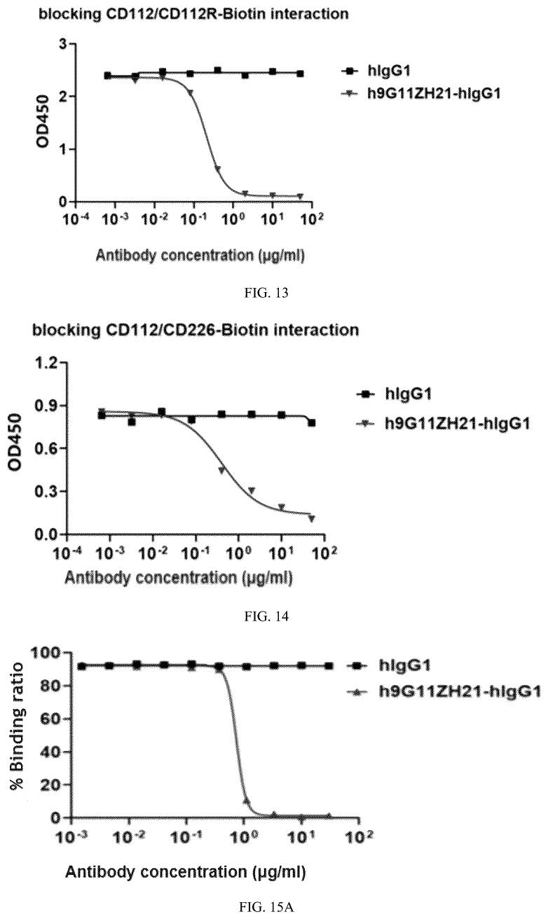

The human CD112-His protein was diluted to 2 μg/ml with PBS buffer and added to a 96-well plate in a volume of 100 μL/well and left overnight at 4° C. The PBS buffer solution was sucked off from the 96-well plate. The plate was washed 6 times with PBST (pH 7.2, PBS containing 0.1 vol % of Tween 20) buffer solution. 200 μL/well of PBS containing 10% BSA was added and incubated at 37° C. for 2 hours for blocking. The blocking solution was removed and the plate was washed 6 times with PBST. 50 μL of CD112R-Fc-Biotin (2 μg/mL) or CD226-Biotin (50 μg/mL) (purchased from Acro) diluted to an appropriate concentration with PBST containing 0.05% BSA was added per well. Then, the wells containing CD112R-Fc-Biotin or CD226-Biotin were added with 50 μL of the affinity mature antibody h9G11ZH21-hIgG1 (heavy chain sequence is SEQ ID NO: 23, and light chain sequence is SEQ ID NO: 24) diluted gradient-wise in a PBST containing 0.05% BSA or the control hIgG1 (purchased from Biointron), respectively, in a total volume of 100 μL, followed by incubation for 1 h at 37° C. The reaction system was removed. The plate was washed 6 times with PBST. 100 μL/well of HRP (horseradish peroxidase)-labeled Streptavidin secondary (purchased from Southern Biotech) antibody diluted with PBST containing 0.05% BSA (diluted at 1:5000) and incubated for 1 h at 37° C. The plate was washed 6 times with PBST, 80 μL/well TMB (tetramethylbenzidine) was added. After 3 min of incubation at room temperature, and 80 μL/well 4M sulfuric acid was added to terminate the reaction. The absorbance was read at 450 mm with a microplate reader. The results are shown in FIGS. 13 and 14, demonstrating that the affinity mature CD112 antibody h9G11ZH21-hIgG1 of the present disclosure can block CD112 from binding to its receptor CD112R and CD226.

### Example 12: Detection of Blocking Ability of Affinity Mature CD112 Antibody (Flow Cytometry Method)

Flow cytometry experiment was used to detect the blocking characteristic of the affinity mature CD112 antibody h9G11ZH21-hIgG1. CD112 antibody was added to CHO-K1-human CD112 cells prepared in Example 10, followed by CD112R-Fc-Biotin or CD226-Biotin protein, and the signal intensity after the addition of Alexa-647-labeled Streptavidin antibody secondary antibody (purchased from Biolegend) was used to determine the binding characteristics of antibody and CD112.

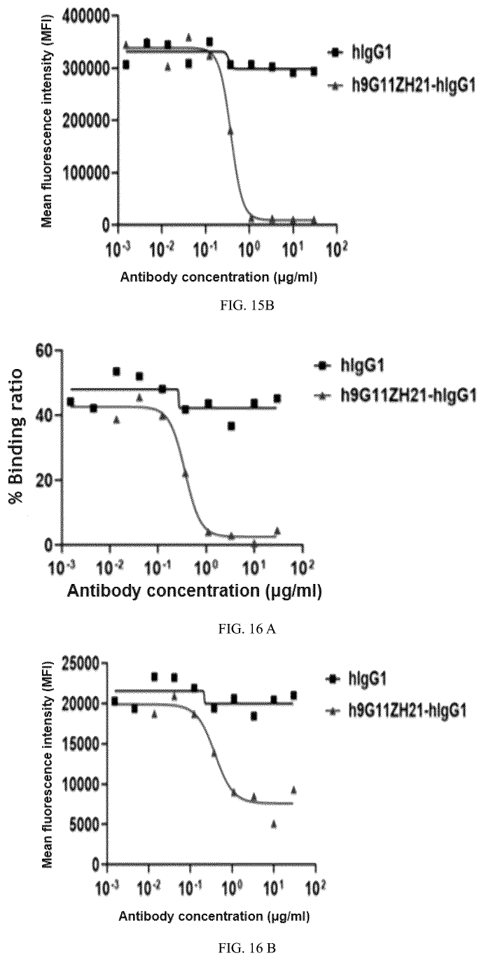

CHO-K1-human CD112 cells were diluted to 2×10⁶/mL with PBS and were added to a 1.5 mL EP tube at 100 μL/tube. Goat serum at 10 μL/tube was added thereto and was blocked at 4° C. for 30 min. The affinity mature antibody h9G11ZH21-hIgG1 (heavy chain sequence is SEQ ID NO: 23, and light chain sequence is SEQ ID NO: 24) and control hIgG1 (purchased from Biointron) diluted gradient-wise (up to 30 μg/ml, 10 gradients, diluted 3-fold for each gradient) were added and incubated at 4° C. for 30 min. CD112R-Fc-Biotin or CD226-Biotin protein was then added and incubated for 30 min at 4° C. The EP tube was added with 1 mL of PBS, and centrifuged at 3500 rpm at 4° C. for 5 min. The supernatant was discarded and the pellet was washed once more with PBS. After centrifugation, the supernatant was discarded. The cells were resuspended with 100 μL/tube of PBS, to which 1 μL/tube of Alexa-647-labeled Streptavidin antibody secondary antibody (purchased from Biolegend) was added, and incubated at 4° C., protected from light, for 30 min. The cell-containing pellet was washed twice with PBS and the supernatant was discarded after centrifugation. The cells were resuspended with 200 μL/tube PBS and detected by the flow cytometry. The results are as shown in FIGS. 15A, 15B, 16A and 16B, further showing that the affinity mature CD112 antibody h9G11ZH21-hIgG1 of the present disclosure can block CD112 from binding to its receptor, CD112R and CD226.

### Example 13: CD112 Antibody Promoting Antigen-Specific T Cell Activation

CMV-specific T cells were co-cultured with HepG2 hepatoma cells, and CMV peptide segment and antibody were added into the culture system. After 24 h of co-culture, IFN-γ secretion in the supernatant was detected to characterize T cell activation.
(1) HepG2 tumor cells were diluted to 1.25×10⁵/mL, and added into a 96-well plate, 80 μl/well;
(2) CMV peptide CMV (purchased from MBL) was added at 20 μL/well to a final concentration of 1 μg/mL and incubated overnight at 37° C. in 5% CO₂;
(3) CMV-specific T cells were diluted to 1.25×10⁶/mL, and added into a 96-well plate, 80 μL/well;
(4) the affinity maturation antibody h9G11ZH21-hIgG1 (heavy chain sequence is SEQ ID NO: 23, and light chain sequence is SEQ ID NO: 24), PD-1 antibody (purchased from Biointron) and control hIgG1 (purchased from Biointron) were added, with a final antibody concentration of 20 μg/mL; and
(5) after 24 h of incubation at 37° C. with 5% CO₂, the culture supernatant was collected, and the content of IFN-γ in the supernatant was determined using the IFN-γ ELISA detection kit (purchased from Dayou).

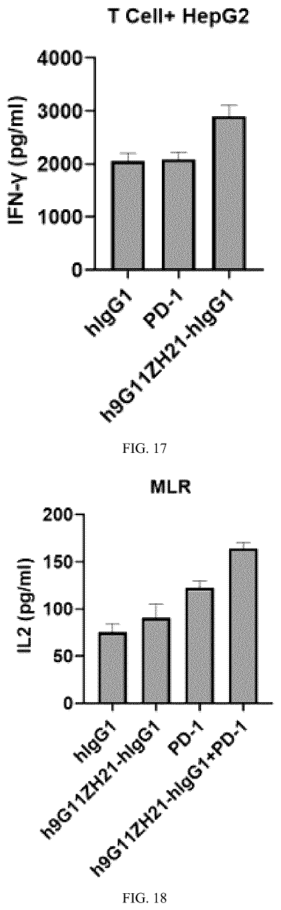

Results are as shown in FIG. 17. The affinity mature CD112 antibody h9G11ZH21-hIgG1 can promote the secretion of IFN-γ by the antigen-specific T cell, whereas the control PD-1 antibody cannot promote the secretion of IFN-γ by the antigen-specific T cell.

### Example 14: CD112 Antibody and PD-1 Antibody Synergistically Promoting T Cell Activation in Mixed Lymphocyte Reaction

CD112 antibody was added into the mixed lymphocyte reaction system, and after 72 h of co-culture, the secretion of IL-2 in the supernatant was detected to characterize T cell activation.
(1) MDDC cells were diluted to 1.25×10⁵/mL, and added into a 96-well plate, 80 μl/well;
(2) the affinity maturation antibody h9G11ZH21-hIgG1 (heavy chain sequence is SEQ ID NO: 23, and light chain sequence is SEQ ID NO: 24), PD-1 antibody (purchased from a Biointron), h9G11ZH21-hIgG1, PD-1 antibody, and control hIgG1 (purchased from Biointron) were added, with a final antibody concentration of 20 μg/mL; and
(3) PBMC cells (purchased from Sailybio) were diluted to 2×10⁶/mL and added to a 96-well plate, 100 μL/well;
(5) after 72 h of incubation at 37° C. with 5% CO₂, the culture supernatant was collected, and the content of IL-2 in the supernatant was determined using IL-2 ELISA detection kit (purchased from Dayou).

Results are shown in FIG. 18. PD-1 antibody can promote the secretion of IL-2 by T cells, while affinity maturation CD112 antibody h9G11ZH21-hIgG1 could not promote the secretion of IL-2 by T cells alone, but h9G11ZG21-hIgG1 could promote the secretion of IL-2 by T cells synergistically with PD-1 antibody, and the effect of promoting the secretion of IL-2 by T cells was stronger than that of PD-1 antibody alone.

### Example 15: CD112 Antibody Promoting Anti-Tumor of NK92 Cell

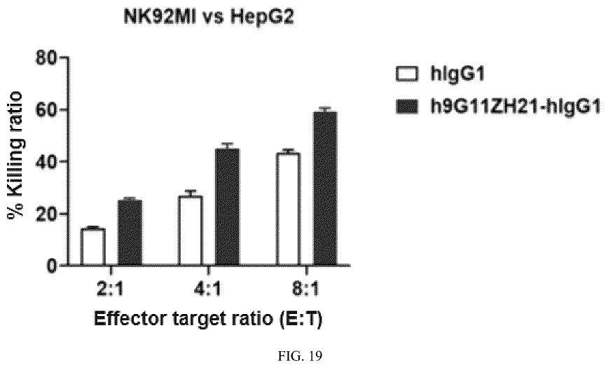

This example is an in vitro killing experiment to detect the effect of affinity mature CD112 antibody h9G11ZH21-hIgG1 on the killing of HepG2 cells by NK92MI cells.
(1) HepG2 tumor cells were washed twice with a serum-free DMEM medium, centrifuged at 4° C. and 200 g for 5 min, and the supernatant was discarded. The resulting pellet was resuspended in 1 mL of serum-free DMEM medium and CTV (purchased from Thermo) with a final concentration of 5 μM was added and incubated at 37° C. for 30 min.
(2) After the incubation, 200 μL of pre-cooled fetal bovine serum was added to the culture system to terminate the labeling, and centrifuged at 4° C. and 200 g for 5 min, and the supernatant was discarded. The cell pellet was washed twice with a complete RPMI-1640 medium containing 10% fetal bovine serum, and the supernatant was discarded. The pellet was resuspended with the complete RPMI-1640 medium, the cells were counted and diluted to 1.25×10⁵ cells/mL with the complete RPMI-1640 medium.
(3) NK92MI cells were diluted to a range of concentrations (2×10⁵/mL, 4×10⁵/mL, 8×10⁵/mL) with the complete RPMI-1640 medium containing 10% fetal bovine serum.
(4) The diluted NK92MI cells were added to a 96-well round bottom plate in a volume of 100 μL/well.
(5) The affinity mature antibody h9G11ZH21-hIgG1 (heavy chain sequence is SEQ ID NO: 23, and light chain sequence is SEQ ID NO: 24) and control hIgG1 (purchased from Biointron) were diluted with complete RPMI-1640 medium and added into a 96-well round bottom plate at 20 μL/well.
(6) The diluted tumor cells were added to the 96-well round bottom plate in a volume of 80 μL/well. The final concentration of antibody is 20 μg/mL.
(7) The 96-well round bottom plate in step (6) was centrifuged at 1500 rpm for 1 min at 4° C., and the centrifuged 96-well plate was incubated at 37° C. for 4 h.
(8) 7-AAD dye (purchased from BD) was added to a 96-well round bottom plate in a volume of 1 μL/well and mixed well. All the liquid in the well was transferred into a flow cytometry tube and detected by using a flow cytometer. The specific experimental results are shown in FIG. 19, indicating that the CD112 antibody of the present disclosure can promote the killing of HepG2 tumors by NK92MI cells.

From the above experimental results, it can be seen that the antibody obtained by the present disclosure can bind to CD112, block the interaction between CD112 and CD112R, TIGIT, and CD226, promote T cell activation, and promote NK cell anti-cancer.

In the present description, descriptions of the reference terms such as “one embodiment”, “some embodiments”, “examples”, “specific examples” and “some examples” mean that, specific features, structures, materials or characteristics described in combination with the embodiments or examples are included in at least one embodiment or example of the present disclosure. In the present description, schematic expressions of the above terms are unnecessarily specified at the same embodiment or example. Moreover, the specific features, structures, materials or characteristics described herein may be in combination in any of or more embodiments or examples in an appropriate mode. In addition, in absence of mutual contradiction, different embodiments or examples described in the present description and features of the different embodiments or examples may be in integration and combination by those skilled in the art.

Although the embodiments of the present disclosure have been illustrated and described above, it can be understood that the above embodiments are illustrative and not limitations on the present disclosure. Those skilled in the art can make changes, modifications, replacements and transformations to the above embodiments without departing from the scope of the present disclosure.

What is claimed is:

1. An antibody or an antigen-binding fragment thereof, comprising light chain variable region CDRs and heavy chain variable region CDRs; wherein:
a heavy chain variable region CDR1 sequence is as set forth in GFTFSSYX₁, where X₁ is Y or M;
a heavy chain variable region CDR2 sequence is as set forth in INSQGGST;
a heavy chain variable region CDR3 sequence is as set forth in ARSDYDWAWX₂AY, where X₂ is F or Y;
a light chain variable region CDR1 sequence is as set forth in QSLLYSSNQKNY;
a light chain variable region CDR2 sequence is as set forth in WAX₃, where X₃ is S or N; and
a light chain variable region CDR3 sequence is as set forth in QQYYRYPPT.

2. The antibody or antigen-binding fragment thereof according to claim 1, wherein the antibody comprises at least one of the following CDR sequences:
heavy chain variable region CDR sequences: SEQ ID NO: 1, SEQ ID NO: 2, SEQ ID NO: 3, SEQ ID NO: 7, and SEQ ID NO: 8; and
light chain variable region CDR sequences: SEQ ID NO: 4, SEQ ID NO: 5, SEQ ID NO: 6, and SEQ ID NO: 9.

3. The antibody or antigen-binding fragment thereof according to claim 1, wherein:
the heavy chain variable region CDR sequence has at least 80% similarity to any one of sequences as set forth in SEQ ID NO: 1 to SEQ ID NO: 3 or SEQ ID NO: 7 to SEQ ID NO: 8; and
the light chain variable region CDR sequence has at least 80% similarity to any one of the sequences as set forth in SEQ ID NO: 4 to SEQ ID NO: 6 or SEQ ID NO: 9.

4. The antibody or antigen-binding fragment thereof according to claim 1, comprising:
the heavy chain variable region CDR1 sequence, the heavy chain variable region CDR2 sequence, the heavy chain variable region CDR3 sequence as set forth in SEQ ID NO: 1, SEQ ID NO: 2, and SEQ ID NO: 3, respectively, or as set forth in amino acid sequences having at least 80% similarity to SEQ ID NO: 1, SEQ ID NO: 2, and SEQ ID NO: 3, respectively; or
the heavy chain variable region CDR1 sequence, the heavy chain variable region CDR2 sequence, the heavy chain variable region CDR3 sequence as set forth in SEQ ID NO: 7, SEQ ID NO: 2, and SEQ ID NO: 8, respectively, or as set forth in amino acid sequences having at least 80% similarity to SEQ ID NO: 7, SEQ ID NO: 2, and SEQ ID NO: 8, respectively.

5. The antibody or antigen-binding fragment thereof according to claim 1, comprising:
the light chain variable region CDR1 sequence, the light chain variable region CDR2 sequence, the light chain variable region CDR3 sequence as set forth in SEQ ID NO: 4, SEQ ID NO: 5, and SEQ ID NO: 6, respectively, or as set forth in amino acid sequences having at least 80% similarity to SEQ ID NO: 4, SEQ ID NO: 5, and SEQ ID NO: 6, respectively; or
the light chain variable region CDR1 sequence, the light chain variable region CDR2 sequence, the light chain variable region CDR3 sequence as set forth in SEQ ID NO: 4, SEQ ID NO: 9, and SEQ ID NO: 6, respectively, or as set forth in amino acid sequences having at least 80% similarity to SEQ ID NO: 4, SEQ ID NO: 9, and SEQ ID NO: 6, respectively.

6. The antibody or antigen-binding fragment thereof according to claim 1, comprising:
at least one of a heavy chain frame region sequence and a light chain frame region sequence,
wherein at least one part of the at least one of the heavy chain frame region sequence and the light chain frame region sequence is derived from at least one of a murine antibody, a humanized antibody, a primate-derived antibody, or a mutant thereof.

7. The antibody or antigen-binding fragment thereof according to claim 1, wherein:
the antibody or antigen-binding fragment thereof comprises a heavy chain variable region having an amino acid sequence as set forth in SEQ ID NO: 10, SEQ ID NO: 12, or SEQ ID NO: 14; and/or,
the antibody or antigen-binding fragment thereof comprises a light chain variable region having an amino acid sequence as set forth in SEQ ID NO: 11, SEQ ID NO: 13, or SEQ ID NO: 15;
optionally, the antibody or the antigen-binding fragment thereof comprises the heavy chain variable region having the amino acid sequence as set forth in SEQ ID NO: 10 and the light chain variable region having the amino acid sequence as set forth in SEQ ID NO: 11;
optionally, the antibody or the antigen-binding fragment thereof comprises the heavy chain variable region having the amino acid sequence as set forth in SEQ ID NO: 12 and the light chain variable region having the amino acid sequence as set forth in SEQ ID NO: 13; and
optionally, the antibody or antigen-binding fragment thereof comprises the heavy chain variable region having the amino acid sequence as set forth in SEQ ID NO: 14 and the light chain variable region having the amino acid sequence as set forth in SEQ ID NO: 15.

8. The antibody or antigen-binding fragment thereof according to claim 1, comprising at least one of a heavy chain constant region and a light chain constant region, wherein at least one part of the at least one of the heavy chain constant region and the light chain constant region is derived from at least one of a humanized antibody, a primate-derived antibody, a murine antibody, or a mutant thereof;
optionally, the light chain constant region and the heavy chain constant region are both derived from:
a murine IgG1 antibody, a murine IgG2a antibody, or a mutant thereof; or
a humanized IgG1 antibody, a humanized IgG2 antibody, a humanized IgG3 antibody, a humanized IgG4 antibody, or a mutant thereof.

9. A nucleic acid molecule, encoding the antibody or antigen-binding fragment thereof according to claim 1.

10. An expression vector, carrying the nucleic acid molecule according to claim 9.

11. A method for preparing the antibody or antigen-binding fragment thereof according to claim 1, the method comprising:
introducing an expression vector into cells, the expression vector carrying a nucleic acid molecule encoding the antibody or antigen-binding fragment thereof; and
culturing the cells under conditions suitable for protein expression and secretion, to obtain the antibody or antigen-binding fragment thereof,
optionally, the cells are prokaryotic cells or eukaryotic cells; and
optionally, the cells are eukaryotic cells.

12. A recombinant cell, expressing the antibody or antigen-binding fragment thereof according to claim 1.

13. A composition, comprising the antibody or antigen-binding fragment thereof according to claim 1.

14. A method for preventing and/or treating a CD112-mediated disease, comprising:
administering the antibody or antigen-binding fragment thereof according to claim 1 to a subject in need thereof.

15. The method according to claim 14, wherein the CD112-mediated disease is transplant rejection, an autoimmune disease, an infectious disease, or a cancer; and
the cancer is selected from at least one of lung cancer, liver cancer, ovarian cancer, cervical cancer, skin cancer, bladder cancer, colon cancer, breast cancer, glioma, renal cancer, gastric cancer, esophageal cancer, oral squamous cell carcinoma, or head-and-neck cancer.

16. A medicament, comprising the antibody or antigen-binding fragment thereof according to claim 1, wherein the medicament is used for preventing and/or treating a CD112-mediated disease.

17. The medicament according to claim 16, wherein the CD112-mediated disease is transplant rejection, an autoimmune disease, an infectious disease, or a cancer; and
the cancer is selected from at least one of lung cancer, liver cancer, ovarian cancer, cervical cancer, skin cancer, bladder cancer, colon cancer, breast cancer, glioma, renal cancer, gastric cancer, esophageal cancer, oral squamous cell carcinoma, or head-and-neck cancer.

18. A kit, comprising the antibody or antigen-binding fragment thereof according to claim 1.
